(19) United States
(12) Patent Application Publication
(10) Pub. No.: US 2024/0367072 A1
(43) Pub. Date: Nov. 7, 2024

(54) PROTEIN A/G PARTICLES FOR AFFINITY CHROMATOGRAPHY AND METHODS OF USE THEREOF

(71) Applicant: Waters Technologies Corporation, Milford, MA (US)

(72) Inventors: Kevin Wyndham, Worcester, MA (US); Beatrice Muriithi, Attleboro, MA (US); Oksana Tchoul, Winchester, MA (US); Yeliz Tunc Sarisozen, Westford, MA (US); Mingcheng Xu, Lexington, MA (US); Matthew Lauber, North Smithfield, RI (US)

(73) Assignee: Waters Technologies Corporation, Milford, MA (US)

(21) Appl. No.: 18/654,384

(22) Filed: May 3, 2024

Related U.S. Application Data
(60) Provisional application No. 63/587,864, filed on Oct. 4, 2023.
(60) Provisional application No. 63/464,384, filed on May 5, 2023.

(51) Int. Cl. B01D 15/38; B01J 20/26; B01J 20/28

(52) U.S. Cl. CPC B01D 15/3809; B01J 20/262; B01J 20/28004; B01J 20/28016

(57) ABSTRACT

The present disclosure is directed to nonporous polymer particles having an average particle size of 1 to 10 micrometers and being functionalized with an immunoglobulin-binding protein. The functionalized particles of the present disclosure can be used to purify a range of immunoglobulins with affinity to the immunoglobulin-binding protein.

### CROSS-REFERENCE TO RELATED APPLICATIONS

This application claims the benefit of U.S. Provisional Application No. 63/464,384 filed on May 5, 2023 and U.S. Provisional Application No. 63/587,864 filed on Oct. 4, 2023, the entireties of which are incorporated herein by reference.

### FIELD OF THE TECHNOLOGY

The present technology is directed to affinity capture materials. More particularly, the present technology is directed to chromatographic columns with nonporous particles for use in affinity chromatography.

### BACKGROUND

Chromatography is a separation technique with broad utility across industries, including the pharmaceutical, biotechnology, and chemical industries. Chromatography involves two phases: a stationary phase and a mobile phase. Typically, the stationary phase comprises a porous or nonporous particles that are loaded into a column. The mobile phase then carries a sample through the stationary phase column. One particular application of chromatography, affinity chromatography, utilizes functionalized particles in the stationary phase. The underlying principle behind affinity chromatography is that compounds, or analytes, within a sample will have differential affinities to the functional group on the particle. These differences permit the separation, isolation, and concentration of particular analytes from complex, heterogeneous samples.

In one instance, the analyte of interest to be isolated using affinity chromatography is an antibody. Antibodies are typically isolated from culture medium (e.g., hybridoma cultures), serum, or ascites fluid, each of which contain an unpredictable number and composition of analytes. But due to the complexity and/or volume of the sample which contains the antibody, the antibody can be at such a low concentration, or in the presence of enough impurities, that it is undetectable using standard analytical methods. Further, the utility of antibodies in experimental and therapeutic applications often requires a homogenous, pure, and concentrated sample. As such, it is highly desirable to be able to isolate an antibody that is both substantially pure and at high enough concentrations that permit downstream use across applicable assays.

Current methods of affinity chromatography are hindered by columns that are low throughput and high cost, and often result in loss of antibody due to non-specific binding and/or poor antibody retention. Other types of immunoassays, such as for example, ELISA technology, can be complex, time consuming and highly variable. Therefore, a need in the art exists for affinity chromatography columns that are highly efficient and enable robust separation, collection, and concentration of a variety of antibodies independent of their target antigen.

### SUMMARY OF THE INVENTION

In general, the present technology is directed to a plurality of particles used in affinity chromatography. More specifically, the present technology is directed to a chromatographic column containing a plurality of particles having an average particle size of less than 10 μm, wherein an outer hydrophilic surface of each of the particles is conjugated to an immunoglobulin-binding protein, such as Protein A, which serves as the binding site for an antibody. As a result, the chromatographic columns of the present technology can be used in affinity chromatography of antibodies.

In one aspect, the present technology is directed to a chromatographic column. The chromatographic column including a column body formed of a metal or metal alloy and housing a plurality of particles. Each particle of the plurality of particles comprising a nonporous polymer core, a hydrophilic surface on an outer layer of the nonporous polymer core; and one or more molecules of an immunoglobulin-binding domain conjugated to the hydrophilic surface, wherein the particle has an average particle size between 1.5 μm to 8 μm.

Other embodiments of the technology are as follows. In some embodiments, the immunoglobulin-binding protein is Protein A, Protein G, Protein A/G, or Protein L, or a binding domain thereof. In some embodiments, the nonporous polymer core has a gradient composition. In some embodiments, the nonporous polymer core comprises divinylbenzene (80%). In some embodiments, the hydrophilic surface is (3-glycidyloxypropyl)trimethoxysilane, (3-glycidyloxypropyl)triethoxysilane, polyacrylate, glycidol, glyceroltri glycidyl ether, butyl diglycidol ether, or poly(methyl acrylate). In certain embodiments, the one or more molecules of immunoglobulin-binding protein are conjugated to the hydrophilic surface of the particle via an epoxy linker. In some embodiments, the average particle size is between 2 to 5 μm. In one embodiment, the average particle size is 3.5 μm. In some embodiments, the immunoglobulin-binding protein has a surface coverage of between 3-9 μg per mg of particle. In some embodiments, the immunoglobulin-binding protein is Protein A and has a surface coverage of between 3-9 μg per mg of particle.

In some embodiments, the chromatographic column has at least a portion of an interior surface of the column body coated with an alkylsilyl material. In some embodiments, the column body comprises frits, wherein the frits are coated with the alkylsilyl material. In some embodiments the alkylsilyl material is a hydrophilic, non-ionic layer of polyethylene glycol silane.

In one aspect, disclosed herein is a chromatographic device comprising the chromatographic column, a column injector positioned upstream of the chromatographic column, and tubing in fluidic connection with and located downstream of the chromatographic column, wherein a portion of an internal surface of the column injector and a portion of an internal surface of the tubing are coated with an alkylsilyl material.

In one aspect, disclosed herein is a method for enriching an immunoglobulin, the method comprising four steps a) through d). Step a) comprises selecting a chromatographic column, wherein the chromatographic column comprises a column body formed of a metal or a metal alloy, the column body housing a plurality of particles, with each particle of the plurality of particles comprising a nonporous polymer core, a hydrophilic surface on an outer layer of the nonporous polymer core, and one or more molecules of an immunoglobulin-binding protein conjugated to the hydrophilic surface, wherein the particle has an average particle size between 1.5 to 8 μm. Step b) comprises washing the chromatographic column with a binding buffer. Step c) comprises applying a solution containing an immunoglobulin to the chromatographic column. Step d) comprises washing the chromatographic column with an elution buffer such that the immunoglobulin is eluted from the chromatographic column.

In some embodiments, the immunoglobulin binding protein is Protein A, Protein G, Protein A/G, Protein L, or a binding domain thereof. In some embodiments, the chromatographic column is connected to a high-performance liquid chromatography (HPLC) system, ultra-high performance liquid chromatography (UHPLC) system, or fast protein liquid chromatography (FPLC) system. In some embodiments, the elution buffer is at least 3 orders of magnitude more acidic than the binding buffer. In some embodiments, the binding buffer has a pH between 7.0 to 8.0. In some embodiments, the elution buffer has a pH between 1.3 and 3.5.

In some embodiments, the method further comprises step e) detecting the immunoglobulin with an ultraviolet spectroscopy detector, a fluorescence spectroscopy detector, and/or a mass spectrometry detector. In some embodiments, the method further comprises step f) washing the chromatographic column with the binding buffer. In some embodiments, the method further comprises repeating steps b) through e).

In other embodiments, the method further comprises step e) washing the chromatographic column with the binding buffer. In some embodiments, the method further comprises repeating steps b) through d).

In some embodiments, the chromatographic column is washed with the binding buffer for less than or equal to 10 minutes. In some embodiments, the method can be performed without the need for an organic modifier to reduce non-specific binding.

In some embodiments, the applying a solution containing an immunoglobulin to the chromatographic column (step c) is performed in about 1 to 3 minutes. In some embodiments, the applying a solution containing an immunoglobulin to the chromatographic column (step c) is performed in about 1 minute.

### BRIEF DESCRIPTION OF THE DRAWINGS

The technology will be more fully understood from the following detailed description taken in conjunction with the accompanying drawings, in which:

FIG. 1A is a cross-sectional illustration of a particle prior to attachment of an immunoglobulin-binding protein in accordance with an embodiment of the technology.

FIG. 1B is a cross-sectional illustration of the particle of FIG. 1A after attachment of an immunoglobulin-binding protein in accordance with an embodiment of the technology.

FIG. 2A is a perspective view of a chromatographic column packed with a plurality of particles (i.e., the particles of FIG. 1B) having a cut out section (220) to illustrate an interior portion of the chromatographic column.

FIG. 2B is a cross-sectional view of the chromatographic column of FIG. 2A taken along line BB.

FIG. 3 is a schematic illustrating a method of performing affinity capture of an antibody with the chromatographic column of FIG. 2B.

FIG. 4A shows the peak area of eluted IgG at different concentrations, between 0.25 and 20 μg, for the column of FIG. 2 (solid black line) as compared to Comparator Column 1 column (dotted line).

FIG. 4B shows the peak area of eluted IgG at different concentrations, between 0.25 and 300 μg, for the column of FIG. 2 (solid black line) as compared to the Comparator Column 1 column (dotted line).

FIG. 4C depicts the percent carry over of IgG between affinity capture assays for the column of FIG. 2 as compared to the Comparator Column 1.

FIG. 5A shows the elution peaks of eluted IgG for the column of FIG. 2 (dashed black line) as compared to the Comparator Column 3 column (dashed line) and the Comparator Column 2 column (solid line).

FIG. 5B shows the percent carry over of IgG between affinity capture assays for the column of FIG. 2 as compared to Comparator Column 2 and Comparator Column 3.

FIG. 6A depicts the peak area of eluted IgG for the column of FIG. 2 using a 1 minute binding phase (solid black line) or a 3 minute binding phase (dashed black line).

FIG. 6B shows the quantified peak area of eluted IgG for the column of FIG. 2 using a 1 minute binding phase or a 3 minute binding phase across different concentrations of IgG.

FIG. 6C depicts the percent carryover of IgG between affinity capture assays for the column of FIG. 2 using a 1 minute binding phase or a 3 minute binding phase across different concentrations of IgG.

FIG. 7A-7C provides chromatographs of monoclonal antibody eluted from a column with particle type B using a phosphoric acid elution method (FIG. 7A) or a glycine elution method (FIG. 7B). FIG. 7C shows the linear correlation between monoclonal antibody concentration and peak area.

FIG. 8A-8C provides chromatographs of monoclonal antibody eluted from a column with particle type D using a phosphoric acid elution method (FIG. 8A) or a glycine elution method (FIG. 8B). FIG. 8C shows the linear correlation between monoclonal antibody concentration and peak area.

FIG. 9A-9C provides chromatographs of monoclonal antibody eluted from a column with particle type H using a phosphoric acid elution method (FIG. 9A) or a glycine elution method (FIG. 9B). FIG. 9C shows the linear correlation between monoclonal antibody concentration and peak area.

FIG. 10A-10C provides chromatographs of monoclonal antibody eluted from a column with particle type E using a phosphoric acid elution method (FIG. 10A) or a glycine elution method (FIG. 10B). FIG. 10C shows the linear correlation between monoclonal antibody concentration and peak area.

FIG. 11A-11C provides chromatographs of monoclonal antibody eluted from a column with particle type C using a phosphoric acid elution method (FIG. 11A) or a glycine elution method (FIG. 11B). FIG. 11C shows the linear correlation between monoclonal antibody concentration and peak area.

FIG. 12A-12C provides chromatographs of monoclonal antibody eluted from a column with particle type F using a phosphoric acid elution method (FIG. 12A) or a glycine elution method (FIG. 12B). FIG. 12C shows the linear correlation between monoclonal antibody concentration and peak area.

FIG. 13A-13C provides chromatographs of monoclonal antibody eluted from a column with particle type A using a phosphoric acid elution method (FIG. 13A) or a glycine elution method (FIG. 13B). FIG. 13C shows the linear correlation between monoclonal antibody concentration and peak area.

FIG. 14A-14B provide chromatographs comparing peak shape for particle types A, B, C, D, E, F, and H using a phosphoric acid elution method (FIG. 14A) or a glycine elution method (FIG. 14B).

FIG. 15A-15B compare the peak height for particle types A, B, C, D, E, F, and G for both elution methods (FIG. 15A) and the peak volume for both elution methods (FIG. 15B).

### DETAILED DESCRIPTION

In order that the technology may be more readily understood, certain terms are first defined. In addition, it should be noted that whenever a value or range of values of a parameter are recited, it is intended that values and ranges intermediate to the recited values are also part of this disclosure. The word “about” if not otherwise defined means ±5%. It is also to be noted that as used herein and in the claims, the singular forms “a,” “and” and “the” include plural references unless the context clearly dictates otherwise.

### Definitions

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

The term “rigid particle,” as used herein, refers to the strength of the particle to withstand applied pressures under flow conditions. A rigid particle appears visually undamaged (i.e., maintains the same form factor without breaking, crushing, or alteration) in a scanning electron microscope image after exposure to pressures of 3,500 psi, wherein less than 10% of the observed particles are visually damaged. In addition, particles in a packed bed that are broken or deformed result in reduced flow and increased pressure as one would predict using the Kozeny-Carmen equation. Broken or deformed particles in a packed bed can increase pressure beyond levels suitable for use in HPLC or UHPLC.

The term “conjugated,” as used herein, refers to the linkage of two molecules formed by the chemical bonding of a reactive functional group of one molecule, such as Protein A, with an appropriately reactive functional group of another molecule, such as an epoxide.

The term “conjugate,” as used herein, refers to a compound formed by the chemical bonding of a reactive functional group of one molecule, such as Protein A, with an appropriately reactive functional group of another molecule, such as an epoxide. An example of suitably reactive functional groups is a nucleophile/electrophile pair. For instance, the nucleophile may be an amine or thiol group from an amino acid of Protein A, and the electrophile is an epoxide.

As used herein, the term “immunoglobulin” and “antibody” are used interchangeably unless defined otherwise.

The term “antibody,” as used herein refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, and heteroconjugate antibodies (e.g., bi-, tri-, and quad-specific antibodies, diabodies). Unless otherwise indicated, the term “monoclonal antibody” is meant to include both intact molecules as well as antibody fragments that are capable of specifically binding to a target protein.

Unless defined otherwise, all technical and scientific terms used herein have the same meaning as commonly understood by one of ordinary skill in the art to which this disclosure belongs.

### Materials

The present technology is directed to materials, columns, and devices used in affinity chromatography. In particular, the present technology is directed to materials for use in a high performance liquid chromatography (HPLC) system or ultra-high performance liquid chromatography (UHPLC) system and are tailored to allow for affinity capture at the high pressures and flow conditions associated with HPLC and/or UHPLC.

In the present technology, a plurality of particles is housed within a chromatographic column. Along with other components, it is these particles that have been designed to provide on-column affinity capture at the high pressures and flow rates associated with HPLC and UHPLC systems.

### Particles

To provide stability and the surface area for affinity capture, the particles of the present technology are nonporous. The nonporous particles provide the appropriate surface area for the attachment or coverage with one or more affinity binding groups. In some embodiments, each particle within the plurality of particles to be packed into a column may be highly spherical and have a smooth surface. In some embodiments, each particle within the plurality of particles to be packed into a column may be highly spherical and have a bumpy convex surface. Such materials have surface areas (measured in m²/g) that are close to their theoretical values. The theoretical surface area for a nonporous smooth sphere is equal to 6/{particle diameter×particle density}. For example, 1 micron polymer particles with a density of approximately 1 g/mL has a theoretical surface area of 6 m²/g, a 3.5 micron polymer particle with the same density has a theoretical surface area of 1.7 m²/g, and a 7 micrometer polymer particle with same density has a theoretical surface area of 0.9 m²/g.

Without wishing to be bound by theory, it is believed that the use of nonporous spheres is advantageous as it improves the kinetics of binding and elution of affinity binding groups attached to the surface of the sphere (having either a smooth or bumpy with convex surfaces). It is believed that the form factor of a nonporous sphere shuts down diffusion kinetics into pores of the particles.

The particles are nonporous. While some pores or porosity may be incorporated within the particles as discontinuities or as microporosity, nonporous particles are those particles having a pore volume that is less than 0.1 cc/g of the material forming the particle. Preferably, nonporous particles have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

The particles of the present technology have an average particle size of less than 10 micrometers. For example, the average particle size of a plurality of particles packed within a column in an embodiment of the present technology can be a value anywhere between 8 micrometers and 1.5 micrometers. In one embodiment, the average particle size of the plurality of particles is 7 micrometers. In another embodiment, the average particle size is 3.5 micrometers. In still yet another embodiment, the average particle size is 1.7 micrometers.

The size (i.e., less than 10 micrometers), shape (i.e., spherical), and surface area (i.e., nonporous, smooth or bumpy convex outer surface) create a form factor useful for affinity capture from a flowing sample. To obtain high throughput and to minimize assay development, the particles of the present technology are used in conjunction with LC systems, such as HPLC and UHPLC systems. These systems operate under high pressures (e.g., typically greater than 3,000 psi, such as, for example, 5,000 psi, 10,000 psi, 12,000 psi, 15,000 psi and so forth). As a result, the particles of the present technology need to be rigid particles, such that the particles retain their form factor under HPLC and UHPLC operating conditions.

In general, the particles of the present technology are rigid particles that maintain their form factors (e.g., are not damaged, crushed, squished, or altered) under HPLC or UHPLC operating conditions (e.g., pressures and flow rates). For example, rigid particles in accordance with the present technology, are not visibly altered in form (e.g., not broken, crushed, or altered from spherical) as can be confirmed using scanning electron microscopy before (i.e., control) and after application of HPLC or UHPLC conditions.

A particular material for forming a core (e.g., center or base) of the particles of the present technology that meets the form factor considerations is polymers, and in particular organic polymers. In an embodiment, the nonporous particles of the present technology include a nonporous polymer core. In one embodiment, the nonporous polymer cores of the particles are divinylbenzene (DVB), for example divinylbenzene 80%. In some embodiments, the nonporous polymer cores are formed to include two or more polymers. For example, in some embodiments the nonporous polymer cores include both divinylbenzene and polystyrene. In certain embodiments, the nonporous polymer cores can be manufactured to include a gradient in the polymer composition. For example, the inner portion of the core can be formed of 100% of first polymer (i.e., polymer A) and an outer portion of the core can be formed of 100% or some percentage greater than 0% of a second polymer (i.e., polymer B). Radially from the inner portion to the outer portion of the core, the percentage of polymer A and polymer B can vary to form the gradient in polymer composition. Other embodiments of nonporous polymer cores and particles suitable for use with the present technology are described in U.S. Patent Publication No. 2019/0322783.

While examples and embodiments of the present technology illustrate the use of nonporous polymer cores for the particles, it is noted that other nonporous materials can be utilized as long as the form factor of the particles can be maintained under the operating conditions of HPLC or UPHLC. That is, other materials, such as silica, metal oxides, hybrid inorganic-organic materials, or combinations thereof may be used to create nonporous spherical particles having an average particle size of less than 10 micrometers and that have the rigidity or strength to retain their form factor under the high operating pressures.

To form particles useful for affinity capture, the outer surface of the nonporous core of the particles is linked or connected to an affinity attachment group. To do so, in one embodiment, the outer surface of the nonporous polymer core contains a hydrophilic material. That is, a hydrophilic surface is created on this outer region of the nonporous polymer core. To the hydrophilic surface, one or more molecules of an immunoglobulin-binding protein is conjugated to the hydrophilic surface. The one or more molecules of the immunoglobulin-binding protein provide accessible binding sites for an immunoglobulin. In some embodiments, the immunoglobulin-binding protein is Protein A, Protein G, Protein A/G, or Protein L. In one aspect, any immunoglobulin-binding protein that binds to a conserved portion of an antibody is suitable for use with the present technology.

The hydrophilic surface can also be referred to as a hydrophilic layer. The hydrophilic surface is located on the outer surface of the nonporous polymer core and can be formed of a polymer, molecule or siloxane that has a high density of hydrophilic groups (e.g., hydroxyls, PEG, sugars or carbohydrates). The immobilization of these hydrophilic groups can occur by condensation (ester, amid, silanol, sily ether), polymerization (methacrylates, acrylates, styryl) epoxy activation (epihydrochlorin), or ether formation (direct attachment of PEG or carbohydrate groups by ether formation).

In one embodiment, the hydrophilic surface comprises a material selected from the group consisting of: (3-glycidyloxypropyl)trimethoxysilane, (3-glycidyloxypropyl)triethoxysilane, polyacrylate, poly(methyl acrylate), and combinations thereof. In another embodiment, the hydrophilic surface comprises a material selected from the group consisting of: glycidol, glyceroltriglycidyl ether, and combinations thereof.

In embodiments in which an immunoglobulin-binding protein is used for providing binding sites for antibodies, a linker is typically used to secure or to conjugate the immunoglobulin-binding protein to the hydrophilic surface. Such linkers include, but are not limited to epoxy linkers, hydroxyl linkers, and any other linkers as are known in the art (see Hermanson G, “Bioconjugate Techniques” 3^{rd} Edition, July 2013).

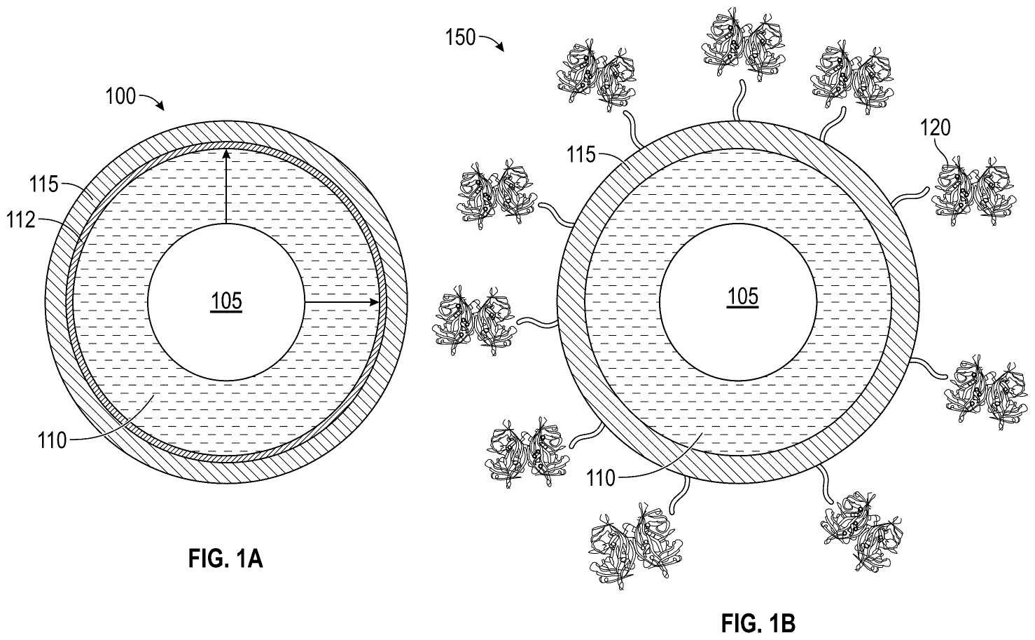

FIG. 1A illustrates an embodiment of a particle having a nonporous polymer core in accordance with the present technology. That is, the particle illustrated in FIG. 1A has a form factor (e.g., spherical, nonporous, and rigid) to withstand operating conditions of HPLC and UHPLC. Particle 100 shown in FIG. 1A is a cross-sectional view prior to the addition of an immunoglobulin-binding protein such as for example Protein A. Particle 100 includes a nonporous polymer core 112 having an inner core region 105 and a radially extending region 110 surrounding the inner core region 105. The inner core region 105 typically is formed of a polymer or a homogenous blend of polymers, whereas the radially extending region 110 typically is formed of two or more polymers to form a gradient within this region. For example, core region 105 can be formed of polystyrene, whereas radially extending region 110 contains a gradient composition transitioning from 100% polystyrene to 80% to 100% DVB with any remainder being polystyrene.

In one embodiment, to form nonporous polymer cores 112, the following three steps were used. In step one: 561.1 g of reagent alcohol (90% ethanol, ˜5% methanol and ˜5% isopropanol), 16.9 g of polyvinylpyrrolidone (PVP-40, average molecular weight 40,000), 1.6 g of 2,2′-Azobis(2-methylpropionitrile) (AIBN), 6.7 g of Triton™ N-57, 80.1 g of styrene and 2.4 g of poly(propylene glycol) dimethacrylate (average molecular weight 560) were charged into a reactor. After purging with nitrogen, the reaction mixture was heated to 70° C. with stirring and was held at 70° C. until the completion of all the reaction steps. In step two: after the step one reaction mixture was held at 70° C. for 3 hours, a solution containing 52.0 g of DVB 80, 24.0 g of styrene, 51.0 g of PVP-40, 1080.4 g of reagent alcohol (90% ethanol, ˜5% methanol and ˜5% isopropanol) and 54.1 g of p-xylene was added to the reaction mixture at a constant flow rate over two hours. In step three: after the completion of solution charge in step two, a primer coating solution containing 31.2 g of glycidyl methacrylate (GMA), 6.2 g of ethylene glycol dimethacrylate (EDMA), 12.9 g of PVP-40 and 381.9 g of reagent alcohol (90% ethanol, ˜5% methanol and ˜5% isopropanol) was added to the reaction mixture at a constant flow rate over 1.5 hours. After the reaction mixture was held at 70° C. for a total of 20 hours, the particles were separated from the reaction slurry by filtration. The particles were then washed with methanol, followed by tetrahydrofuran (THF), and followed by acetone. The final product was dried in vacuum oven at 45° C. overnight. 91.8 g of monodisperse 2.3 μm polymer particles were obtained.

While the embodiment shown in FIG. 1A illustrates that the nonporous polymer core 112 has two regions (the inner core region 105 and the radially extending region 110), that need not be the case. Other embodiments may feature a nonporous polymer core having a singular region, i.e., the nonporous polymer core extends from the center of the particle to an outer surface of the nonporous polymer core 112.

As illustrated in FIG. 1A, a hydrophilic surface or layer 115 is formed on an outer surface (i.e., opposite to the center region 105) of the nonporous polymer core. In one embodiment, the hydrophilic surface 115 is formed through the application of a hydrophilic primer coating solution containing 36.2 g of glycidyl methacrylate (GMA), 7.44 g of ethylene glycol dimethacrylate (EDMA), 8.21 g of PVP360 (PVP360, average molecular weight 360,000) and 489.4 g of reagent alcohol (90% ethanol, ˜5% methanol and ˜5% isopropanol). This solution was added into to a mixture containing the nonporous polymer cores at a constant flow rate over about 1.5 hours to form hydrophilic surface 115.

The above example is provided for illustration purposes only. Other types of hydrophilic surfaces can be applied. For example, the hydrophilic layer may also be formed of (3-glycidyloxypropyl)trimethoxysilane, (3-glycidyloxypropyl)triethoxysilane, polyacrylate, and/or poly(methyl acrylate), glycidol, glyceroltriglycidyl ether, butyl diglycidol ether, or any other type of hydrophilic material.

To attach the immunoglobulin-binding protein, e.g., Protein A, a linker is used to conjugate it the hydrophilic surface 115. Referring to FIG. 1B, shown is a particle 150 after conjugation. That is, particle 150 is the result of conjugating the immunoglobulin-binding protein 120 to the hydrophilic surface 115 through use of the linker. As described above any type of linker can be used. One of ordinary skill in the art would understand there are various ways to attach the immunoglobulin-binding protein to the hydrophilic layer. A number of methods for conjugation are suitable for use with the present technology. For example, an epoxy linker can be used to join the immunoglobulin-binding protein to the hydrophilic group 115.

The immunoglobulin-binding protein provides accessible binding sites for an immunoglobulin, i.e., an antibody, provided that said antibody comprises a conserved region that binds to the immunoglobulin-binding protein. In one embodiment, the immunoglobulin-binding protein is Protein A. In another embodiment, the immunoglobulin-binding protein is Protein G. In other embodiments, the immunoglobulin-binding protein is Protein A/G or Protein L.

Most immunoglobulins (Ig) consist of four polypeptide chains: two identical heavy chains and two identical light chains that are connected by disulfide bonds. Within a given heavy chain or light chain, there is both a variable and a constant region. The constant region, which comprises 2-4 constant domains (depending on isotype), is highly conserved within a given isotype. As such, immunoglobulin-binding proteins that bind to a portion of the constant region are suitable for affinity capture of antibodies independent of the antibody's target antigen.

Typically, immunoglobulin-binding proteins bind to the fragment crystallizable (Fc) region of an antibody, which comprises a portion of the heavy chain constant domains of the antibody. In at least one instance, an immunoglobulin-binding protein has been shown to bind instead to a constant domain of the light chain.

Immunoglobulin-binding proteins suitable for use in the present technology may exhibit strong binding affinity to the Fc portion of an antibody. This binding affinity can vary in strength by both isotype and species. For example, Protein A exhibits strong binding affinity to IgG isotypes but variable to no binding affinity to IgA, IgD, IgE, and IgM isotypes. Even within the IgG isotype, different subclasses can exhibit varied binding affinity. Protein A has high binding affinity to human IgG1, IgG2, and IgG4, but very weak binding affinity to IgG3. By contrast, Protein A binds to murine IgG3 but not to IgG1. Other examples of immunoglobulin-binding proteins, such as Protein G, have high binding affinity to all four subclasses of IgG. Methods for characterizing protein-protein interactions, including binding affinities across a range of environmental conditions, are well known in the art.

The present technology enables the conjugation of any immunoglobulin-binding protein to the hydrophilic surface of the nonporous polymer particle. As such, it is within the scope of the technology that the immunoglobulin-binding protein may bind to IgA, IgD, IgE, IgG, and/or IgM antibodies with varying levels of affinity. In some embodiments, the immunoglobulin-binding protein is selected from the group consisting of: Protein A, Protein G, Protein A/G, and Protein L. In some embodiments, the immunoglobulin-binding protein may be a variant of Protein A, Protein G, Protein A/G, or Protein L. In some embodiments, the immunoglobulin-binding protein may have 80-85%, 85-90%, 90-95%, or 95-100% sequence identity to Protein A, Protein G, Protein A/G, or Protein L.

In some embodiments, the immunoglobulin-binding protein has a surface coverage of between 3-9 μg per mg of particle. In some embodiments, the immunoglobulin-binding protein has a surface coverage of between 3-3.5 μg per mg of particle, 3.5-4.0 μg per mg of particle, 4.0-4.5 μg per mg of particle, 4.5-5 μg per mg of particle, 5-5.5 μg per mg of particle, 5.5-6 μg per mg of particle, 6-6.5 μg per mg of particle, 6.5-7 μg per mg of particle, 7-7.5 μg per mg of particle, 7.5-8 μg per mg of particle, 8-8.5 μg per mg of particle, or 8.5-9 μg per mg of particle. In some embodiments, the immunoglobulin-binding protein is Protein A and has a surface coverage of between 3-9 μg per mg of particle. In some embodiments, the immunoglobulin-binding protein is Protein G and has a surface coverage of between 3-9 μg per mg of particle. In some embodiments, the immunoglobulin-binding protein is Protein A/G and has a surface coverage of between 3-9 μg per mg of particle. In some embodiments, the immunoglobulin-binding protein is Protein L and has a surface coverage of between 3-9 μg per mg of particle.

Examples 1 and 2 provide exemplary methods of synthesizing nonporous polymer particles that can be functionalized with an immunoglobulin-binding protein, such as, for example, Protein A, Protein G, Protein A/G, or Protein L.

Without being bound by any particular theory, it is understood that the binding affinity of the antibody to the immunoglobulin-binding protein is dependent at least in part on the pH of the environment. As such, the binding of the antibody to the immunoglobulin-binding protein is reversible by altering the pH of the buffer within the column of the present technology.

### Columns and Devices

In the present technology, the materials described above are typically packed into a chromatographic device, such as, for example a chromatographic column. The chromatographic device includes a column body formed of a metal or a metal alloy, e.g., titanium, stainless steel. The column body houses a plurality of the particles, such as a plurality of the particle shown in FIG. 1B.

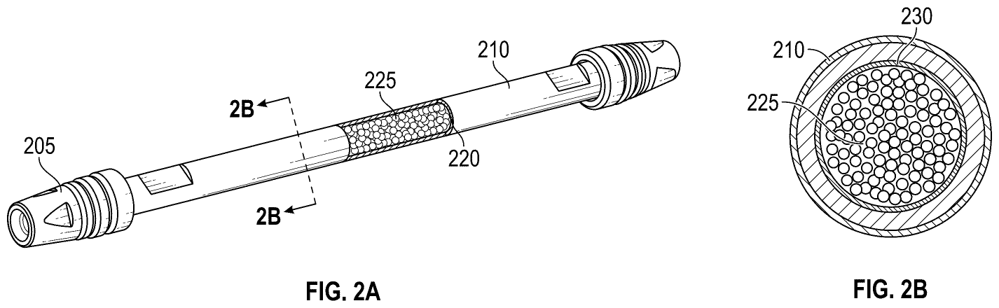

Referring to FIG. 2A, a chromatographic column 205 is shown. This chromatographic column 205 has a stainless steel column body 210. A portion 220 of the column body is removed in FIG. 2A to illustrate the location of a plurality of particles 225. FIG. 2B provides a cross-sectional view of the chromatographic column 205 taken along line B-B in FIG. 2A.

The cross-sectional view of FIG. 2B illustrates the position of the column body 210 surrounding and housing the plurality of particles 225. In some embodiments, an alkylsilyl coating or other high performance surface is provided to limit or reduce non-specific binding of a sample with the walls or interior surfaces 230 of the column body 210. Without wishing to be bound by theory, it is believed that an alkylsilyl coating covering metal surfaces prevent or minimize contact between fluids passing through the column body 210 and the interior surfaces 230. The alkylsilyl coating can be applied to the interior surfaces 230 of the metal column body 210 defining what is known as a wetted path of the column. A metal wetted path includes all surfaces formed from metal that are exposed to fluids during operation of the chromatographic column. The metal wetted path includes not only the column body walls but also metal frits disposed within the column. In embodiments, the alkylsilyl coating is applied not only to the walls of the column body 210, but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors (e.g., tubing and connectors leading from the column to downstream components such as detectors). Further, the affinity chromatographic columns of the present technology do not require the addition of additional organic modifiers to reduce non-specific binding. Typically, the addition of an organic modifier (e.g., acetonitrile) may be necessary with sorbents used in affinity chromatography to reduce non-specific binding. Due to the already low non-specific binding of the columns of the present technology, no organic modifier is necessary.

In one embodiment, the alkylsilyl coating comprises a hydrophilic, non-ionic layer of polyethylene glycol silane. In another embodiment, the alkylsilyl coating is formed from one or more of the following precursor materials bis(trichlorosilyl) ethane or bis(trimethoxysilyl) ethane. Other embodiments of alkylsilyl coatings suitable for use with the present technology are described in US Patent Publication No. 2019/0086371 and US Application Publication No. 2022/0118443.

The chromatographic device of the present technology can be appropriately sized for use in HPLC systems, UHPLC systems, or in FPLC (fast protein liquid chromatography) systems. For example, in an embodiment in which the plurality of particles packed into the column have an average particle size of 3.5 micrometers or 1.7 micrometers, the column body of the present technology can have an internal diameter between 1 mm to 4.6 mm and a column length of between 5 and 50 cm. In certain embodiment, the column body has an internal diameter of between 1 mm and 2.1 mm and a length between 15 and 50 cm.

### Methods of Using Affinity Chromatography Columns

The resultant affinity chromatographic column of the present technology can be used to perform affinity capture of antibodies that bind to the particular immunoglobulin-binding protein conjugated to the particle of FIG. 1B. The affinity chromatographic column is suitable for use in conjunction with any liquid chromatography system as described above. In some embodiments, the liquid chromatography system is an HPLC, UHPLC, or FPLC system.

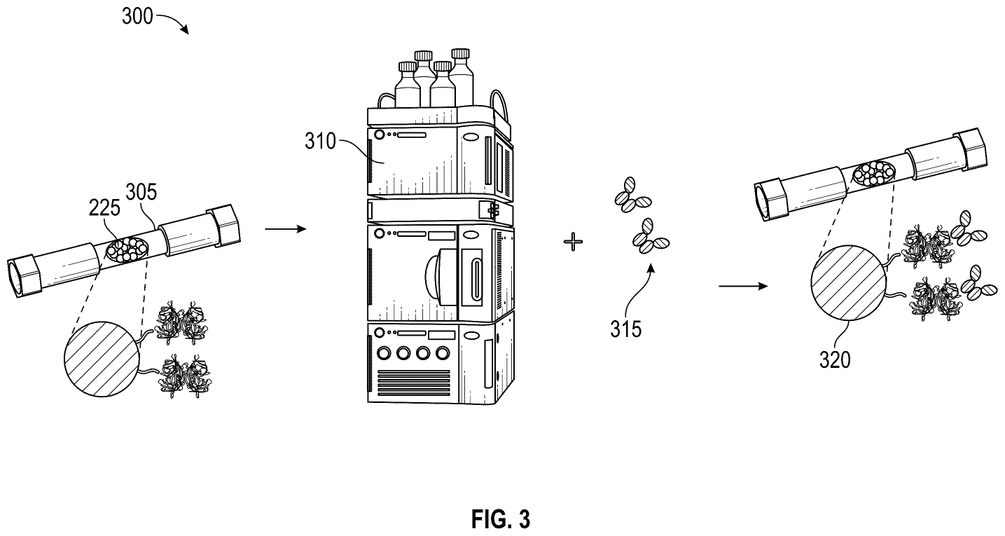

FIG. 3 provides an overview of the method (300) for performing affinity capture of an antibody using the chromatographic column (305) with a plurality of particles functionalized with an immunoglobulin-binding protein (225). The column (305) is connected to a liquid chromatography system (310) and a solution containing an antibody (315) is flowed onto the column. Due to the binding affinity of the antibody to the immunoglobulin-binding protein conjugated to the particles (320), the antibody is immobilized on the column. This can be reversed by changing the environment such that the binding affinity is reduced, for example by making the pH acidic. In doing so, the antibody is no longer immobilized and would elute from the column.

The examples described below use three Comparator Columns, referred to herein and throughout as Comparator Column 1, Comparator Column 2, and Comparator Column 3.

Comparator Column 1 is a polymer-based, Protein A monolith in a 5.6×5 mm stainless steel column.

Comparator Column 2 comprises Protein A conjugated to 12 micron, nonporous polystyrene/divinylbenzene (PS/DVB) particles in a 4×35 mm, polyether ether ketone column.

Comparator Column 3 comprises Protein A conjugated to 20 micron, fully porous (500-1000 Angstrom pores) polystyrene/divinylbenzene (PS/DVB) particles in a 2.1×30 mm polyether ether ketone column.

Example 3 describes a method of performing an affinity capture assay using an affinity chromatographic column prepared with Protein A-functionalized particles. The column was used to bind and elute purified IgG at a range of concentrations. This was then compared to the Comparator Column 1 using similar conditions and samples.

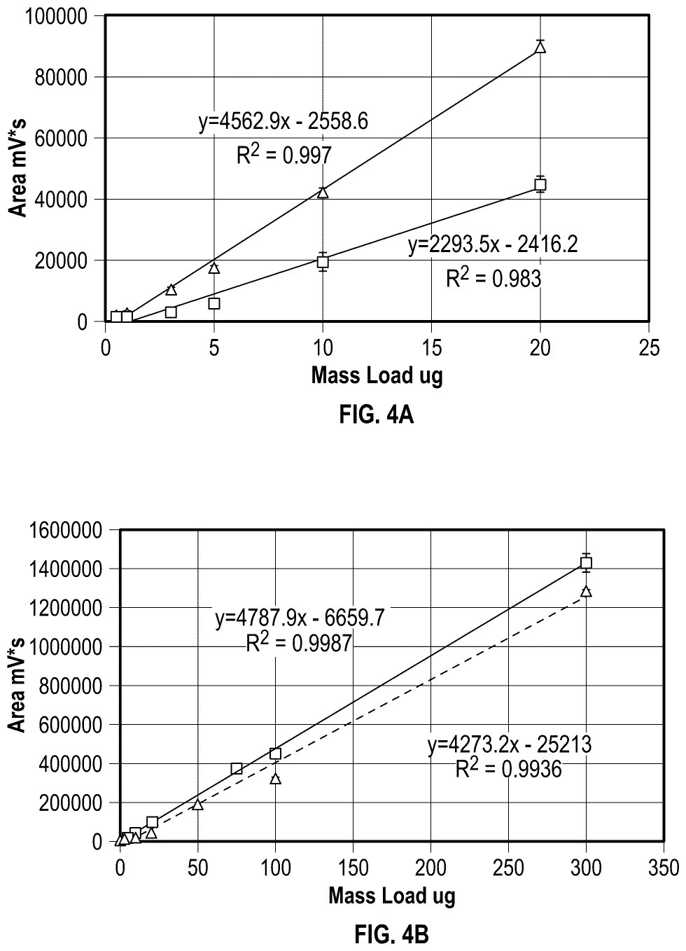

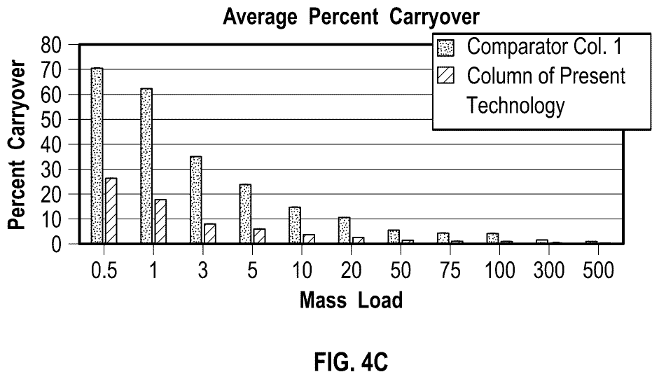

FIG. 4A shows a comparison of the peak volumes for the experiments described in Example 3 for IgG between 0.25 and 20 μg. FIG. 4B shows a comparison of the peak volumes for the experiments described in Example 3 for IgG between 0.25 and 300 μg. The affinity chromatographic column of the present technology provided better peak volumes and enabled more robust titration calculations as compared to Comparator Column 1.

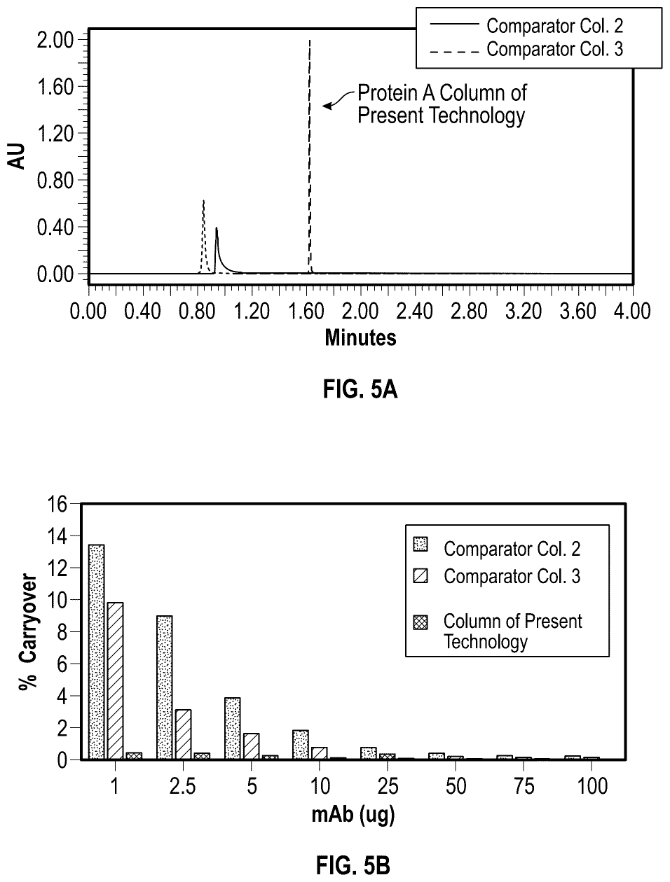

Example 4 describes method of performing an affinity capture assay using an affinity chromatographic column prepared with Protein A-functionalized particles. The column was used to bind and elute purified IgG at a range of concentrations. This was then compared to Comparator Columns 2 and 3 using similar samples and vendor recommended protocols. FIG. 5A demonstrates that the columns of the present technology affords significantly improved peak shape and width relative to the comparator columns. FIG. 5B shows that the columns of the present technology result in significantly reduced percent carryover between affinity capture cycles. The affinity chromatographic column of the present technology provided better peak volumes and enabled more robust titration calculations relative to the comparator columns. The columns of the present technology resulted in >5× increase in sensitivity, reaching detector saturation at injections of 25 μg of IgG due to the enhanced sensitivity of the column.

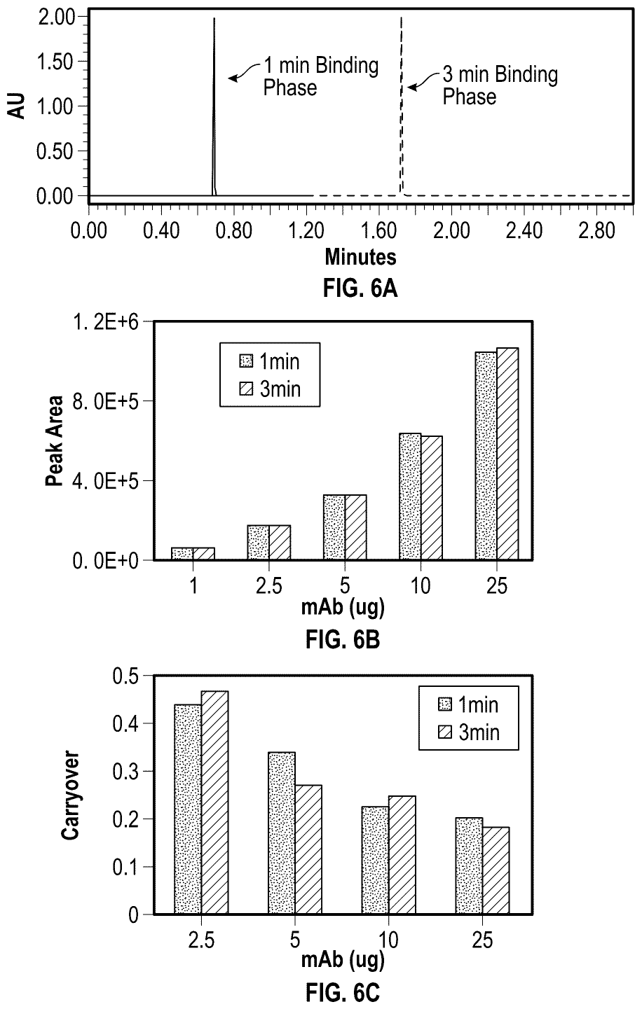

Further, as described in Example 5, the columns of the present technology allow for faster affinity capture of IgG than those available in the art, without sacrificing on sensitivity or performance. FIG. 6A shows that the columns of the present technology allow for the affinity capture of IgG using a 1 minute binding phase, which resulted in comparable peak area (FIG. 6B) and carryover (FIG. 6C) as the 3 minute binding phase. As such, the columns of the present technology allow for the fast determination of IgG titer using binding phase times of about 1 minute.

Buffers suitable for use in liquid chromatography are well known in the art. It is understood that a range of binding buffers are suitable for use with the disclosed technology and a person of ordinary skill in the art could determine without undue experimentation the appropriate binding buffer that is suitable with a given antibody or sample. In one aspect, the binding buffer has a pH between 7.0 and 8.0. In some embodiments, the pH of the binding buffer is between 7.0-7.1, 7.1-7.2, 7.2-7.3, 7.3-7.4, 7.4-7.5, 7.5-7.6, 7.6-7.7, 7.7-7.8, 7.8-7.9, or 7.9-8.0. In one aspect, the elution buffer has a pH between 1.0 and 3.5. In some embodiments, the pH of the elution buffer is between 1.0-1.2, 1.2-1.4, 1.4-1.6, 1.6-1.8, 1.8-2.0, 2.0-2.2, 2.2-2.4, 2.4-2.6, 2.6-2.8, 3.0-3.2, 3.2-3.4, or 3.4-3.5. It is important that the pH of the elution buffer be at least 3 magnitudes more acidic than the binding buffer.

In some embodiments, the binding buffer comprises 20-100 mM sodium phosphate with 125 mM NaCl (pH 7.4). In some embodiments, the binding buffer comprises 100 mM sodium phosphate buffer (pH 7.4). In some embodiments, the binding buffer comprises 20-200 mM ammonium acetate (pH 7.0 to 8.0). In some embodiments, the binding buffer comprises 20-200 mM ammonium formate (pH 7.0 to 8.0). In some embodiments, the binding buffer is compatible with mass spectrometry methods.

In some embodiments, the elution buffer comprises 20-50 mM sodium phosphate (pH 2.3). In some embodiments, the elution buffer comprises 20-50 mM sodium phosphate with 100-500 mM NaCl (pH 2.3). In some embodiments, the elution buffer comprises 6 mM hydrochloric acid (pH 2.5). In some embodiments, the elution buffer comprises 24 mM phosphoric acid (pH 1.93 to 2.05). In some embodiments, the elution buffer comprises 100 mM glycine (pH 2.5). In some embodiments, the elution buffer is 0.1% to 2% acetic acid in water (pH 1.0 to 3.0). In some embodiments, the elution buffer is 0.1% formic acid in water (pH 1.0 to 3.0). In some embodiments, the elution buffer is compatible with mass spectrometry methods.

In some embodiments, the target analyte is eluted from the column using a step elution, wherein the mobile phase is switched from the binding buffer to the elution buffer. In some embodiments, the target analyte is eluted from the column using a gradient elution, wherein the binding buffer is transitioned to the elution buffer as a gradient.

In one aspect, the affinity chromatographic columns of the present technology afford the elution of an immunoglobulin with a small peak volume. In some embodiments, the peak volume is less than 50 μL. In some embodiments, the peak volume is less than 40 μL, less than 30 μL, or less than 20 μL. In some embodiments, the peak volume is less than 10 μL.

In one aspect, the affinity chromatographic columns of the present technology are able to be re-used for affinity capture assays. In this regard, after the elution of the antibody from the column, the column is then re-equilibrated. Re-equilibration involves flowing buffer through the column such that any residual material from the previous assay is removed. In some embodiments, re-equilibration is performed with the binding buffer. In some embodiments, re-equilibration is performed with an equilibration buffer. The equilibration buffer may have a pH that is equal to or lower than the binding buffer. Due to the relationship between pH and protein conformation stability and folding, it may be desirable to immediately neutralize the eluted sample with a base.

In one aspect, the present technology allows for rapid re-equilibration of the affinity chromatographic column. In some embodiments, the affinity chromatographic column is re-equilibrated for 1-10 minutes, 1-2, 2-3, 3-4, 4-5, 5-6, 6-7, 7-8, 8-9, or 9-10 minutes. In some embodiments, it may be required that the chromatographic column is re-equilibrated for longer periods of time.

Following re-equilibration, the affinity chromatographic column is ready to be re-used for affinity capture assays. Affinity chromatographic columns of the instant technology can be re-used 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, or 200 or more times without degradation or noticeable loss of specificity. In one aspect, the affinity chromatographic column of the instant technology can be stored in-between uses. Buffers suitable for the storage of affinity chromatographic columns are well known in the art.

In one aspect, the affinity chromatographic columns of the present technology result in significantly reduced carryover of applied sample following the re-equilibration process.

FIG. 4C and FIG. 5B show that the affinity chromatographic columns of the present technology resulted in 2-5× less carryover between uses as compared to Comparator Columns 1, 2, and 3.

### Methods and Examples

### Example 1: Addition of Epoxy Linker to Hydrophilic Particles

The nonporous, epoxy-modified hydrophilic particles for use in the disclosed methods were prepared as follows. As a first step, 1500 g of reagent alcohol (90% ethanol, ˜5% methanol, and ˜5% isopropanol), 45.1 g of polyvinylpyrrolidone (PVP-40), 4.8 g of 2,2′-Azobis(2-methylpropionitrile), 5.9 g of Triton™ N-57, 81.7 g of styrene were charged into a reactor. After the reactor was purged with nitrogen gas, the reaction mixture was heated to and maintained at 70° C. with stirring for 3 hours.

After three hours, a solution containing 110.4 g of divinylbenzene 80 (DVB), 39.7 g of PVP-40, 510 g of reagent alcohol, and 100.2 g of p-xylene was added to the reaction mixture at a constant flow rate over two hours. Following this step, a primer coating solution containing 26.0 g of glycidyl methacrylate (GMA), 26.0 g of ethylene glycol dimethacrylate (EDMA), 36.4 g of PVP-40, and 560 g of reagent alcohol were added to the reaction mixture at a constant flow rate over 1.5 hours.

The reaction mixture was maintained at 70° C. for a total of 20 hours, after which the particles were separated from the reaction slurry by filtration. The particles were then washed sequentially with methanol, tetrahydrofuran (THF), and acetone. The final product was dried in a vacuum oven at 45° C., resulting in monodisperse 3.5 μm polymer particles. These particles contain a gradient polystyrene/DVB core with a poly(GMA/EDMA) primer. While the above reaction conditions generate 3.5 μm polymer particles, it is understood that particles ranging in sizes from 1.5 μm to 8 μm are within the scope of the disclosure. By altering the concentrations of PVP-40, 2,2′-Azobis(2-methylpropionitrile), and Triton N-57, one of ordinary skill in the art could generate a range of particle sizes.

The resultant 3.5 μm, polystyrene/DVB particles with the poly(GMA/EDMA) primer were then coated with a hydrophilic layer. 70 g of the particles were hydrolyzed in 0.5M H₂SO₄ at 60° C. for 1-20 hours. The hydrolyzed particles were washed sequentially with MilliQ water and methanol, and then dried under vacuum at 45° C. overnight. The dried particles were added into a 1 L three-necked round bottom flask with an overhead stirring motor, stirring shaft, and stir blade, a water cooled condenser, a nitrogen inlet, and a probe-controlled heating mantle. 700 mL of anhydrous diglyme (diethylene glycol dimethyl ether) was added, the flask sealed, and purged with nitrogen for 15 minutes with moderate stirring. 2.0 g of potassium tert-butoxide was added, and the reaction was raised to 70° C. To generate the hydrophilic layer, a mixture of 10.5 g glycidol, 2.6 g of glyceroltriglycidyl ether, and 14.9 g of anhydrous diglyme was prepared separately and added to the particle mixture in four equal aliquots in 30 minute intervals. The reaction was held at 70° C. for 20 hours, cooled to RT, and filtered. The resulting particles were washed sequentially with water 6 times, methanol 3 times, and then dried under vacuum overnight at 45° C. The following procedure results in a hydrophilic layer that is 2-4% (by weight) of the entire particle.

20 g of the resultant 3.5 μm particles with the hydrophilic coating were added to a mixture of 100 g of ethylene glycol diglycidyl ether (EGDGE) and 100 g of MeOH at room temperature. 1 mL of 50% sodium hydroxide in water was added and the reaction was stirred continuously for 20 h. The particles were isolated by filtration, washed with 400 mL of MeOH ten times, and partially dried under nitrogen flow. The particles were stored for later use in a methanol wet bed at 4° C. The resultant particles have sufficient epoxide content to enable functionalization of the particle surface.

### Example 2: Protein A Functionalized Particles

Particles were prepared as described in Example 1 and functionalized with Protein A. 1.5 g of particles were mixed in 8.3 mL of a 50 mM sodium phosphate buffer (pH 8). To this, 0.3 mL of a 50 mg/mL solution of Protein A (15 mg) was added. Next, 21.4 mL of a sodium phosphate/ammonium sulfate solution was added dropwise. The reaction was then stirred for 20 hours between 24-37° C. The final concentration of ammonium sulfate and the temperature of the reaction can be adjusted to manipulate the extent of Protein A coverage on a given particle.

Following the 20 h incubation, 1 g of ethanolamine in 5 mL of sodium phosphate buffer was added and the reaction stirred at RT for 3 hours. Particles were then isolated by filtration and washed sequentially three times with water (pH 4, adjusted with HCl), twice with water, and twice with storage buffer (20 mM PBS, pH 7.3, 0.02% sodium azide). The particles were stored in a sealed container as a slurry in storage buffer (˜10 mL buffer/g of particle) at 4° C. Protein A coverage of the particles was determined using a standard bicinchoninic acid assay (BCA). Max surface coverage of Protein A on the particles was determined to be 4.2-5.0 μg of Protein A per mg of particle. An exemplary Protein A particle has a surface coverage of about 4.8 μg Protein A per mg of particle (Particle Type A).

Alternatively, particles prepared as described in Example 1 can be functionalized with Protein G. 1.5 g of particles are mixed in 8.3 mL of 50 mM sodium phosphate buffer (pH 8). To this, 0.3 mL of a 50 mg/mL solution of Protein G (15 mg) can be added. Next 21.4 mL of a sodium phosphate/ammonium sulfate solution can be added dropwise and the reaction stirred for 20 hours between 24-37° C. The final concentration of ammonium sulfate and the temperature of the reaction can be adjusted to manipulate the extent of Protein G coverage on a given particle. After the 20 h incubation, 1 g of ethanolamine in 5 mL of sodium phosphate buffer can be added and the reaction stirred at RT for 3 hours. Particles can be isolated by filtration and washed sequentially three times with water (pH 4, adjusted with HCl), twice with water, and twice with a storage buffer (such as 20 mM PBS, pH 7.3, 0.02% sodium azide). Protein G coverage of the particles can be determined using methods known in the art, such as a standard BCA assay.

Alternatively, particles prepared as described in Example 1 can be functionalized with Protein A/G. 1.5 g of particles are mixed in 8.3 mL of 50 mM sodium phosphate buffer (pH 8). To this, 0.3 mL of a 50 mg/mL solution of Protein A/G (15 mg) can be added. Next 21.4 mL of a sodium phosphate/ammonium sulfate solution can be added dropwise and the reaction stirred for 20 hours between 24-37° C. The final concentration of ammonium sulfate and the temperature of the reaction can be adjusted to manipulate the extent of Protein A/G coverage on a given particle. After the 20 h incubation, 1 g of ethanolamine in 5 mL of sodium phosphate buffer can be added and the reaction stirred at RT for 3 hours. Particles can be isolated by filtration and washed sequentially three times with water (pH 4, adjusted with HCl), twice with water, and twice with a storage buffer (such as 20 mM PBS, pH 7.3, 0.02% sodium azide). Protein A/G coverage of the particles can be determined using methods known in the art, such as a standard BCA assay.

Alternatively, particles prepared as described in Example 1 can be functionalized with Protein L. 1.5 g of particles are mixed in 8.3 mL of 50 mM sodium phosphate buffer (pH 8). To this, 0.3 mL of a 50 mg/mL solution of Protein L (15 mg) can be added. Next 21.4 mL of a sodium phosphate/ammonium sulfate solution can be added dropwise and the reaction stirred for 20 hours between 24-37° C. The final concentration of ammonium sulfate and the temperature of the reaction can be adjusted to manipulate the extent of Protein L coverage on a given particle. After the 20 h incubation, 1 g of ethanolamine in 5 mL of sodium phosphate buffer can be added and the reaction stirred at RT for 3 hours. Particles can be isolated by filtration and washed sequentially three times with water (pH 4, adjusted with HCl), twice with water, and twice with a storage buffer (such as 20 mM PBS, pH 7.3, 0.02% sodium azide). Protein L coverage of the particles can be determined using methods known in the art, such as a standard BCA assay.

### Example 3: IgG Binding on the Protein A Affinity Chromatographic Column

The particles as prepared in Example 2 were used to perform affinity capture of IgG. The Protein A-functionalized particles were packed in a 2.1×20 mm hardware. This was connected to a liquid chromatography instrument and equilibrated with 20 mM sodium phosphate (pH 7.4) and 125 mM sodium chloride for 5 minutes at 1 mL/min flow rate.

Once equilibrated, a 10 L injection of 10 μg/mL IgG was injected onto the column and flowed for 0.4 minutes at a 2 mL/min flow rate to allow for binding. The column effluent was monitored using a UV detector (280 nm). After the 0.4 binding phase, the elution buffer (20 mM sodium phosphate (pH 2.7)) was flowed onto the column at a 2 mL/min flow rate for 0.4 minutes. Following this the column was re-equilibrated with 20 mM sodium phosphate (pH 7.4) and 125 mM sodium chloride for 0.4 minutes at a 2 mL/min flow rate.

After re-equilibrating the column, a 5 μL injection of 10 μg/mL IgG was injected onto the column, and the binding, elution, and equilibration processes were repeated as described above. This was repeated using volumes resulting in IgG total concentrations of 0.5, 1, 3, 5, 10, 20, 50, 75, 100, 300, and 500 μg. As a comparison, the process was performed using the Comparator Column 1 under similar conditions.

The Protein A column of the present technology (solid black line) resulted in improved peak area relative to Comparator Column 1 (dotted line) (FIG. 4A-B), and further allowed for robust antibody titer calculations. The Protein A column of the present technology resulted in significantly reduced (2-3× less) carryover of target antibody following the elution and equilibration steps as compared to Comparator Column 1 (FIG. 4C).

### Example 4: IgG Binding on the Protein A Affinity Chromatographic Column

The particles as prepared in Example 2 were used to perform affinity capture of IgG. The Protein A-functionalized particles were packed in a 2.1×20 mm hardware. This was connected to a liquid chromatography instrument and equilibrated with binding buffer (100 mM sodium phosphate (pH 7.4) for 10 minutes at 1 mL/min flow rate. The binding buffer can range from 20-100 mM sodium phosphate (pH 7.4) with either 125 mM sodium chloride or 100 mM sodium phosphate. As a comparator, Comparator Column 3 (also referred to herein as Particle Type G) and Comparator Column 2 were used. Comparator Column 3 columns were equilibrated with 20-100 mM sodium phosphate (pH 7.4) with 120-150 mM NaCl. Comparator Column 2 columns were equilibrated with 50 mM sodium phosphate (pH 7.4), 150 mM NaCl, and 5% acetonitrile.

Once equilibrated, a 10 μL injection of 10 μg/mL IgG was injected onto the column and flowed for 1 minute at a 1 mL/min flow rate to allow for binding. The column effluent was monitored using a UV detector (280 nm). After the 1 minute binding phase, the elution buffer (24 mM phosphoric acid (pH 1.93) was flowed onto the column at a 1 mL/min flow rate for 1 minute. Following this the column was re-equilibrated with 100 mM sodium phosphate (pH 7.4) for 10 minutes at a 1 mL/min flow rate.

After re-equilibrating the column, a 10 μL injection of different concentrations of IgG was injected onto the column, and the binding, elution, and equilibration processes were repeated as described above. This process was performed using different concentrations of IgG resulting in IgG total concentrations of 0.5, 1, 2.5, 5, 10 and 25 μg. IgG concentrations with amounts >25 μg resulted in UV detector saturations due to the increased sensitivity of the protein A column.

As a comparison, the process was performed using Comparator Columns 2 and 3 with similar samples. For Comparator Column 3 column, 10 uL of 10 ug/mL IgG was injected onto the column and flowed for 0.6 min at a flow rate of 1 mL/min. Column effluent was monitored using a UV detector (280 nm). After the binding phase, elution buffer (50 mM sodium phosphate, pH 2.3) was flowed onto the column at 1 mL/min for 1.2 min. The column was re-equilibrated with the binding buffer for 2.4 min. For Comparator Column 2 column, 10 uL of 10 ug/mL IgG was injected onto the column and flowed for 0.6 min at a flow rate of 1 mL/min. Column effluent was monitored using a UV detector (280 nm). After the binding phase, elution buffer (50 mM sodium phosphate, pH 2.3, 150 mM NaCl, 5% acetonitrile) was flowed onto the column at 1 mL/min for 1.2 min. The column was re-equilibrated with the binding buffer for 2.4 min. This was repeated using injection volumes resulting in total IgG amounts for 0.5, 1, 2.5, 5, 10, 25, 50, 75, and 100 ug IgG. Unlike the columns of the present technology, no UV detector saturation was observed with Comparator Columns 2 and 3 even at the highest concentration tested.

The Protein A column of the present technology (dashed black line) resulted in improved peak shape and width (i.e., peak area) relative to the Comparator Column 3 (dashed line) and Comparator Column 2 (solid black line) (FIG. 5A), and further allowed for robust antibody titer calculations. The Protein A column of the present technology resulted in significantly reduced (one order of magnitude) carryover of target antibody following the elution and equilibration steps relative to the comparator columns (FIG. 5B). The Protein A column of the present technology further resulted in significantly increased sensitivity (>5×) relative to comparator columns and an order of magnitude decrease in peak width following the elution an equilibration steps as compared to Comparator Column 3.

### Example 5: Protein A Column Affords Fast Affinity Capture of IgG

The particles as prepared in Example 2 were used to perform affinity capture of IgG. The Protein A-functionalized particles were packed in a 2.1×20 mm hardware. This was connected to a liquid chromatography instrument and equilibrated with binding buffer (100 mM sodium phosphate (pH 7.4)) at a 0.5 mL/min flow rate for 10 minutes. Once equilibrated, a 10 μL injection of 10 μg/μL IgG was injected on the column and flowed for 1 minute at a 1 ml/min flow rate to allow for binding. The column effluent was monitored using a UV detector (280 nm). The captured IgG was eluted with elution buffer (100 mM glycine (pH 2.5)) flowed onto the column at a 1 mL/min flow rate for 0.5 min. Following this, the column was re-equilibrated with binding buffer flowed for 0.5 min at a 1 mL/min flow rate. After re-equilibrating the column, a 10 μL injection of different concentrations of IgG was injected onto the column, repeating the binding, elution, and equilibration processes as described above. This was performed using concentrations resulting in IgG total amounts of 0.5, 1, 2.5, 5, 10, and 25 μg. As a comparison, these procedures were also repeated using a binding phase wherein the IgG was injected on the column and flowed for 3 minutes at a 1 mL/min flow rate to allow for binding.

As shown in FIG. 6A, both the 1 minute (solid black line) and 3 minute (dashed black line) binding phases resulted in similar peak width and sensitivity. As shown in FIG. 6B-6C, peak area (FIG. 6B) and carryover (FIG. 6C) were similar between the different binding phase times tested. Accordingly, the Protein A column of the present technology can efficiently capture IgG using a 1 minute binding phase.

### Example 6: Additional Protein A-Functionalized Materials

Nonporous Silica Particles with a Hybrid Coating, a Diol Coating, and a Hydrophilic Coating Particle Type B

Silica-based nonporous, epoxy-modified hydrophilic particles were prepared. Nonporous 3.6 micron silica particles were heat treated (900° C.) in air for 10 hours. The surface of the particles was re-hydroxylated using 10% v/v nitric acid at 100° C. for 16 hours. The reaction was then cooled below 40° C. and the particles were isolated via filtration. The product was washed with water until the pH of the filtrate was greater than 5, after which it was washed with acetone 3 times. Isolated particles were dried in a vacuum oven at 80° C. overnight.

The hydroxylated silica particles were coated with 1,2-bis(triethoxysilane) ethane (BTEE) with tetraethyl orthosilicate (TEOS). A silane reagent was first prepared from the incomplete (˜68%) hydrolytic condensation of BTEE with tetraethyl orthosilicate (TEOS). Ethanol (3.1 mol ethanol/mol silane), TEOS (1:4 molar ratio with BTEE) and 0.1M HCl (19.7 g/mol silane reagent) were added to the BTEE. The solution was heated at 70° C. for 18 h under inert atmosphere. The reaction was increased to 90° C. to remove the ethanol. The reaction was further increased to 100° C. for 1 hour under an inert atmosphere. The mixture was cooled to room temperature (RT) to obtain the condensation product.

Silica particles were fully dispersed in toluene (21 mL/g of particles). Residual water was removed from the material by azeotropic distillation (110° C. for 1 hour). The reaction was held at 40° C. while the silane reagent (0.821 g/g of particle) was added and stirred for 10 minutes. Catalytic aqueous NH₄OH was added (0.05 g/g particle), stirred for an additional 10 minutes at 40° C., and an additional 2 hours at 60° C. The reaction was cooled to RT and particles were isolated via filtration. The particles were washed twice with ethanol (10 mL/g) then dispersed in 70:30 water:ethanol (v/v) at 10 mL/g particle. Ammonium hydroxide solution (1 g/g particle) was added and the mixture stirred for 2 hours at 50° C. The reaction was cooled below 40° C. and the particles were isolated via filtration. Isolated particles were washed twice with 1:1 methanol:water (v/v) at 10 mL/g particle and twice with methanol. The surface modified particles were dried at 70° C. for 16 hours under vacuum.

To ensure uniformity of the hybrid coating layer, modified particles were exposed to elevated temperatures (100-140° C.) and elevated pH (8-9.8) following the hydrothermal treatment process according to methods described in U.S. Pat. Nos. 6,686,035, 7,223,473, and 7,919,177, and International Patent Publication No. WO 2008/103423, each of which are incorporated herein by reference.

The modified particles were dispersed in 1M HCl (8.4 mL/g particle) and the mixture was stirred at 100° C. for 20 hours. The reaction was cooled below 40° C. and the particles were isolated via filtration. Isolated particles were washed with water until the pH of the filtrate was higher than 5, after which the particles were washed with acetone 3 times. The isolated, surface modified particles were dried under vacuum at 70° C. for 16 hours.

The resultant 3.6 micron particles were next coated with glycidoxypropyltrimethoxy silane (GPTMS). 20 mM sodium acetate buffer (pH 5.5) at ˜5 mL/gram particles) was added to a round bottom flask equipped with a thermometer, condenser, and mechanical stirring apparatus. The solution was heated to 70° C., after which GPTMS was added to the flask at a concentration of 10.22 μmol/m² and mixed at temperature. After 1 hour, particles were added to the flask (1 g particle/5 mL of buffer solution). The mixture was stirred at 70° C. for 20 hours, after which the reaction was cooled below 40° C. and filtered. The product was washed 3× with water and transferred to a new flask equipped with a thermometer, condenser, and mechanical stirring apparatus. Acetic acid (0.1M) was added to the flask (5 mL/gram particle) and the slurry was heated to 70° C. for 20 hours. The flask was cooled below 40° C. and filtered. The product was washed with water until the pH of the supernatant increased to above 5 and then washed 3× with methanol. Isolated particles were dried in a vacuum oven at 70° C. for 16 hours.

10 grams of the resultant particles were added into a 1 L three-necked round bottom flask with overhead stirring motor, stirring shift, stir blade, a water-cooled condenser, a nitrogen inlet, and a probe-controlled heating mantle. 100 mL of anhydrous diglyme (diethylene glycol dimethyl ether) was added. The flask was sealed and purged with nitrogen for 15 minutes with moderate stirring. 0.45 g of potassium tert-butoxide was added and the reaction was raised to 70° C. To generate the hydrophilic layer, a mixture of 1.5 g glycidol, 0.4 g glyceroltriglycidyl ether, and 2.2 g of anhydrous diglyme was prepared separately and added to the particle mixture in 4 equal aliquots using 30-minute intervals. The reaction was held at 70° C. for 20 hours, cooled to RT, and filtered. The resultant particles were washed sequentially 6× with water, 3× with methanol, and dried under vacuum overnight at 45° C. The procedure resulted in a hydrophilic layer that is 2-4% by weight of the entire particle.

5 g of the resultant 3.6 micron particles with the hydrophilic coating were added to a mixture of 25 g of ethylene glycol diglycidyl ether (EGDGE) and 25 g of methanol at RT. 0.25 mL of 50% sodium hydroxide in water was added and the reaction was stirred continuously for 20 hours. The particles were isolated by filtration, washed 10× with methanol, and partially dried under nitrogen flow. Particles were stored in a methanol wet bed at 4° C. The resultant particles have sufficient epoxide content to enable functionalized of the particle surface.

Particles were functionalized with Protein A as described in Example 2, resulting in approximately 8.21 ug of Protein A per mg of particle.

The particles were tested for the ability to bind and elute a monoclonal antibody (NISTmAb Standard, available from Sigma-Aldrich). Particles were packed in a 2.1×200 mm column body and connected to an ultra-high performance liquid chromatography system (Acquity Premier available from Waters Technologies Corporation, Milford MA). The resultant column was fast flushed with water (pH 4) to remove unbound protein A at 0.5 mL/min for 10 minutes. The column was then equilibrated in 0.1M sodium phosphate (pH 7.5) at 0.5 mL/min for 10 minutes. 10 uL injections of the monoclonal antibody standard at different concentrations (ranging from 0.001 to 1 ug/uL) was injected onto the column using 0.1M sodium phosphate (pH 7.4) buffer at a flow rate of 1 mL/min. A blank injection of binding buffer was used between samples to calculate carryover. Elution was performed with either 24 mM phosphoric acid (pH 2) or 100 mM glycine (pH 2.4) for 1 minute. The column was equilibrated for 1 minute using 0.1M sodium phosphate buffer (pH 7.4).

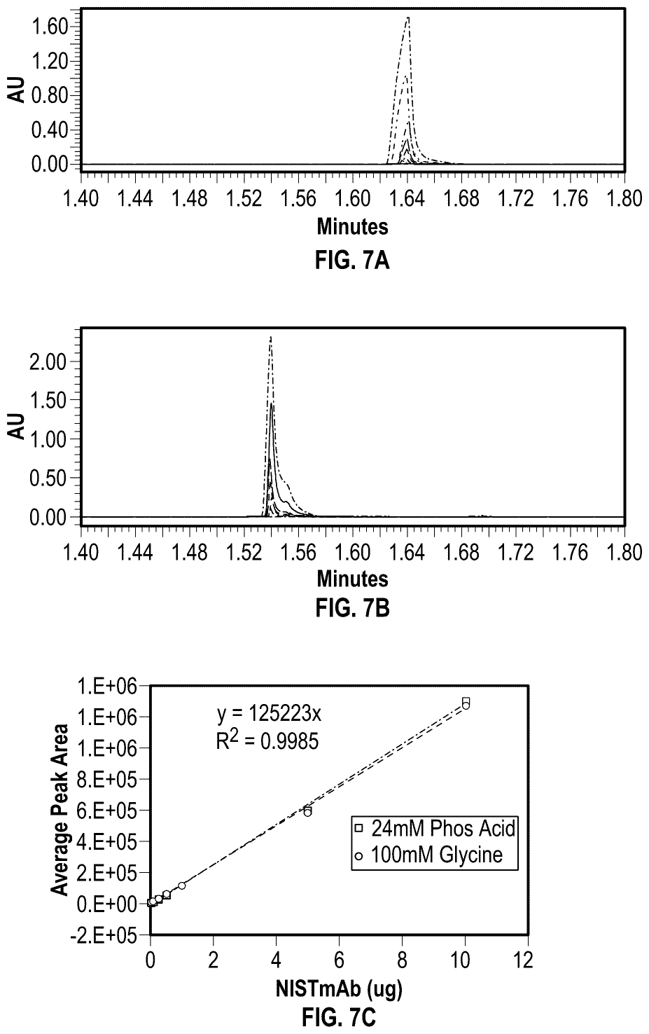

Poor peak shape, fronting, and shouldering was observed due to protein A leaching from the column as shown in FIG. 7A (24 mM phosphoric acid (pH 2)) and FIG. 7B (100 mM glycine (pH 2.4)). There was significant peak tailing for both conditions due to non-specific binding of the monoclonal antibody sample. Thus, even with coating of the silica surface with hybrid material, diol, and a hydrophilic layer, high non-specific binding due to the silica particle was still observed. A low detection amount of 0.025 ug and 0.05 ug was observed for the phosphoric acid and glycine elution buffers, respectively. At low concentrations, >40% carryover was observed due to protein A leaching. At higher concentrations, >6% carryover was observed. FIG. 7C shows the linear correlation between monoclonal antibody concentration and peak area for both elution methods.

Nonporous Silica Particles with a Hybrid Coating and a Diol Coating (Particle Type D)

Silica-based nonporous, epoxy-modified hydrophilic particles were prepared. Nonporous 3.6 micron silica particles were heat treated (900° C.) in air for 10 hours. The surface of the particles was re-hydroxylated using 10% v/v nitric acid at 100° C. for 16 hours. The reaction was then cooled below 40° C. and the particles were isolated via filtration. The product was washed with water until the pH of the filtrate was greater than 5, after which it was washed with acetone 3 times. Isolated particles were dried in a vacuum oven at 80° C. overnight.

The hydroxylated silica particles were coated with 1,2-bis(triethoxysilane) ethane (BTEE) with tetraethyl orthosilicate (TEOS). A silane reagent was first prepared from the incomplete (˜68%) hydrolytic condensation of BTEE with tetraethyl orthosilicate (TEOS). Ethanol (3.1 mol ethanol/mol silane), TEOS (1:4 molar ratio with BTEE) and 0.1M HCl (19.7 g/mol silane reagent) were added to the BTEE. The solution was heated at 70° C. for 18 h under inert atmosphere. The reaction was increased to 90° C. to remove the ethanol. The reaction was further increased to 100° C. for 1 hour under an inert atmosphere. The mixture was cooled to room temperature (RT) to obtain the condensation product.

Silica particles were fully dispersed in toluene (21 mL/g of particles). Residual water was removed from the material by azeotropic distillation (110° C. for 1 hour). The reaction was held at 40° C. while the silane reagent (0.821 g/g of particle) was added and stirred for 10 minutes. Catalytic aqueous NH₄OH was added (0.05 g/g particle), stirred for an additional 10 minutes at 40° C., and an additional 2 hours at 60° C. The reaction was cooled to RT and particles were isolated via filtration. The particles were washed twice with ethanol (10 mL/g) then dispersed in 70:30 water:ethanol (v/v) at 10 mL/g particle. Ammonium hydroxide solution (1 g/g particle) was added and the mixture stirred for 2 hours at 50° C. The reaction was cooled below 40° C. and the particles were isolated via filtration. Isolated particles were washed twice with 1:1 methanol:water (v/v) at 10 mL/g particle and twice with methanol. The surface modified particles were dried at 70° C. for 16 hours under vacuum.

To ensure uniformity of the hybrid coating layer, modified particles were exposed to elevated temperatures (100-140° C.) and elevated pH (8-9.8) following the hydrothermal treatment process according to methods described in U.S. Pat. Nos. 6,686,035, 7,223,473, and 7,919,177, and International Patent Publication No. WO 2008/103423, each of which are incorporated herein by reference.

The modified particles were dispersed in 1M HCl (8.4 mL/g particle) and the mixture was stirred at 100° C. for 20 hours. The reaction was cooled below 40° C. and the particles were isolated via filtration. Isolated particles were washed with water until the pH of the filtrate was higher than 5, after which the particles were washed with acetone 3 times. The isolated, surface modified particles were dried under vacuum at 70° C. for 16 hours.

The resultant 3.6 micron particles were next coated with glycidoxypropyltrimethoxy silane (GPTMS). 20 mM sodium acetate buffer (pH 5.5) at ˜5 mL/gram particles) was added to a round bottom flask equipped with a thermometer, condenser, and mechanical stirring apparatus. The solution was heated to 70° C., after which GPTMS was added to the flask at a concentration of 10.22 μmol/m² and mixed at temperature. After 1 hour, particles were added to the flask (1 g particle/5 mL of buffer solution). The mixture was stirred at 70° C. for 20 hours, after which the reaction was cooled below 40° C. and filtered. The product was washed 3× with water and transferred to a new flask equipped with a thermometer, condenser, and mechanical stirring apparatus. Acetic acid (0.1M) was added to the flask (5 mL/gram particle) and the slurry was heated to 70° C. for 20 hours. The flask was cooled below 40° C. and filtered. The product was washed with water until the pH of the supernatant increased to above 5 and then washed 3× with methanol. Isolated particles were dried in a vacuum oven at 70° C. for 16 hours.

5 g of the resultant 3.6 micron particles with the hydrophilic coating were added to a mixture of 25 g of ethylene glycol diglycidyl ether (EGDGE) and 25 g of methanol at RT. 0.25 mL of 50% sodium hydroxide in water was added and the reaction was stirred continuously for 20 hours. The particles were isolated by filtration, washed 10× with methanol, and partially dried under nitrogen flow. Particles were stored in a methanol wet bed at 4° C. The resultant particles have sufficient epoxide content to enable functionalized of the particle surface.

Particles were functionalized with Protein A as described in Example 2, resulting in approximately 1.84 ug of Protein A per mg of particle.

The particles were tested for the ability to bind and elute a monoclonal antibody (NISTmAb Standard, available from Sigma-Aldrich). Particles were packed in a 2.1×200 mm column body and connected to an ultra-high performance liquid chromatography system (Acquity Premier available from Waters Technologies Corporation, Milford MA). The column was fast flushed with water (pH 4) to remove unbound protein A at 0.5 mL/min for 10 minutes. The column was then equilibrated in 0.1M sodium phosphate (pH 7.5) at 0.5 mL/min for 10 minutes. 10 uL injections of the monoclonal antibody standard at different concentrations (ranging from 0.001 to 1 ug/uL) was injected onto the column using 0.1M sodium phosphate (pH 7.4) buffer at a flow rate of 1 mL/min. A blank injection of binding buffer was used between samples to calculate carryover. Elution was performed with either 24 mM phosphoric acid (pH 2) or 100 mM glycine (pH 2.4) for 1 minute. The column was equilibrated for 1 minute using 0.1M sodium phosphate buffer (pH 7.4).

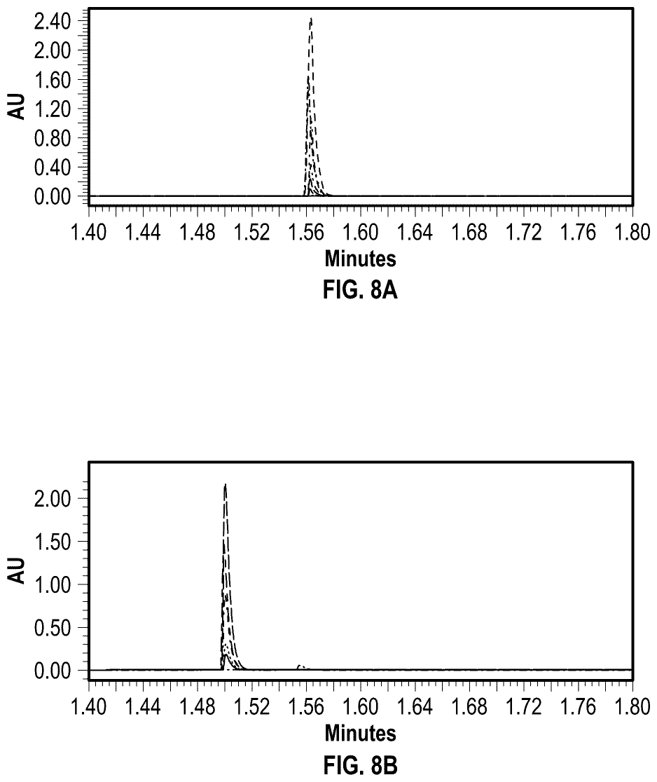

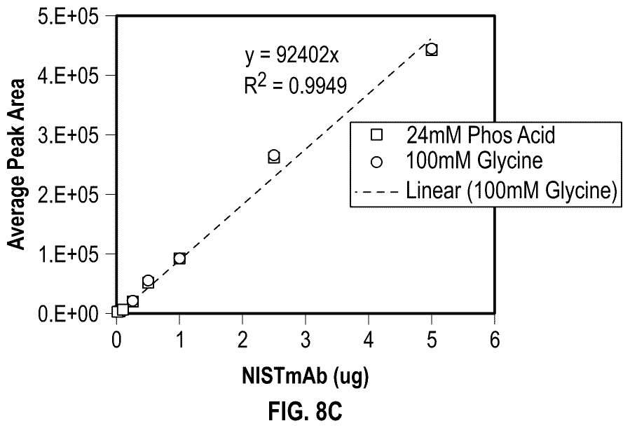

The detection limit was 0.01 ug for the phosphoric acid elution method and 0.25 ug for the glycine elution method. The retention time was shifted in the glycine elution method (see FIG. 8B) as compared to the phosphoric acid elution method (see FIG. 8A). The max pressure that could be applied was 2950 psi. FIG. 8C shows the linear correlation between monoclonal antibody concentration and average peak area for both elution methods. Carryover was less than 3% for glycine elution and <22% for the phosphoric acid elution. The lack of hydrophilic layer results in tailing due to nonspecific binding of the monoclonal antibody on the particle surface.

Nonporous Silica Particles with a Diol Coating (Particle Type H)

Silica-based nonporous, epoxy-modified hydrophilic particles were prepared. Nonporous 3.6 micron silica particles were heat treated (900° C.) in air for 10 hours. The surface of the particles was re-hydroxylated using 10% v/v nitric acid at 100° C. for 16 hours. The reaction was then cooled below 40° C. and the particles were isolated via filtration. The product was washed with water until the pH of the filtrate was greater than 5, after which it was washed with acetone 3 times. Isolated particles were dried in a vacuum oven at 80° C. overnight.

The resultant 3.6 micron particles were next coated with glycidoxypropyltrimethoxy silane (GPTMS). 20 mM sodium acetate buffer (pH 5.5) at ˜5 mL/gram particles) was added to a round bottom flask equipped with a thermometer, condenser, and mechanical stirring apparatus. The solution was heated to 70° C., after which GPTMS was added to the flask at a concentration of 10.22 μmol/m² and mixed at temperature. After 1 hour, particles were added to the flask (1 g particle/5 mL of buffer solution). The mixture was stirred at 70° C. for 20 hours, after which the reaction was cooled below 40° C. and filtered. The product was washed 3× with water and transferred to a new flask equipped with a thermometer, condenser, and mechanical stirring apparatus. Acetic acid (0.1M) was added to the flask (5 mL/gram particle) and the slurry was heated to 70° C. for 20 hours. The flask was cooled below 40° C. and filtered. The product was washed with water until the pH of the supernatant increased to above 5 and then washed 3× with methanol. Isolated particles were dried in a vacuum oven at 70° C. for 16 hours.

5 g of the resultant 3.6 micron particles with the hydrophilic coating were added to a mixture of 25 g of ethylene glycol diglycidyl ether (EGDGE) and 25 g of methanol at RT. 0.25 mL of 50% sodium hydroxide in water was added and the reaction was stirred continuously for 20 hours. The particles were isolated by filtration, washed 10× with methanol, and partially dried under nitrogen flow. Particles were stored in a methanol wet bed at 4° C. The resultant particles have sufficient epoxide content to enable functionalized of the particle surface.

Particles were functionalized with Protein A as described in Example 2, resulting in approximately 2.7 ug of Protein A per mg of particle.

The particles were tested for the ability to bind and elute a monoclonal antibody (NISTmAb Standard, available from Sigma-Aldrich). Particles were packed in a 2.1×200 mm column body and connected to an ultra-high performance liquid chromatography system (Acquity Premier available from Waters Technologies Corporation, Milford MA). The column was fast flushed with water (pH 4) to remove unbound protein A at 0.5 mL/min for 10 minutes. The column was then equilibrated in 0.1M sodium phosphate (pH 7.5) at 0.5 mL/min for 10 minutes. 10 uL injections of the monoclonal antibody standard at different concentrations (ranging from 0.001 to 1 ug/uL) was injected onto the column using 0.1M sodium phosphate (pH 7.4) buffer at a flow rate of 1 mL/min. A blank injection of binding buffer was used between samples to calculate carryover. Elution was performed with either 24 mM phosphoric acid (pH 2) or 100 mM glycine (pH 2.4) for 1 minute. The column was equilibrated for 1 minute using 0.1M sodium phosphate buffer (pH 7.4).

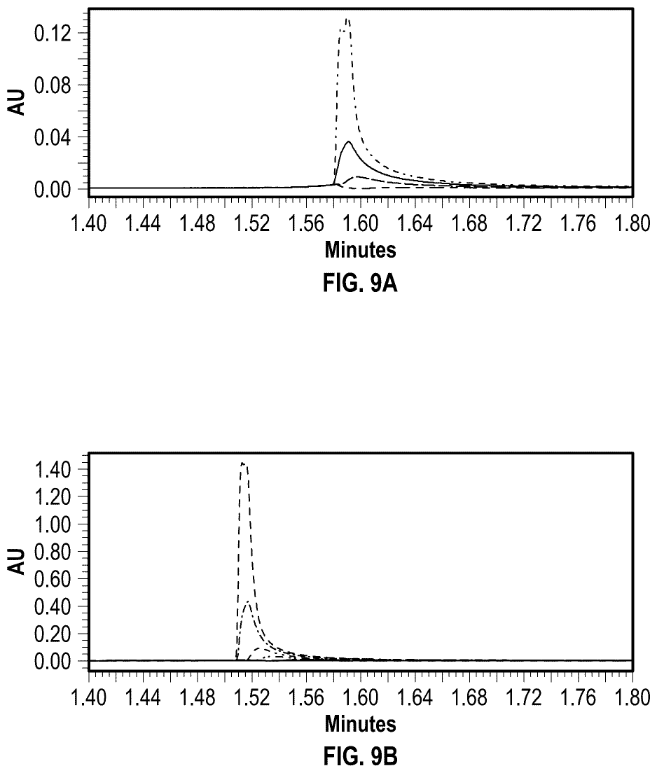

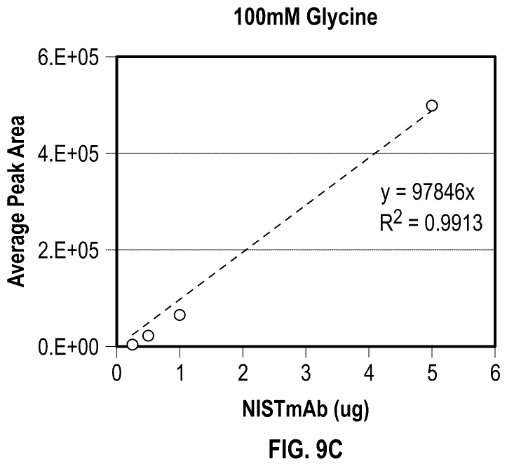

Significant peak tailing was observed for both elution methods as shown in FIG. 9A (24 mM phosphoric acid (pH 2)) and FIG. 9B (100 mM glycine (pH 2.4)). Peak splitting was observed from 0.25 ug/uL for phosphoric acid elution and from 1 ug/uL for the glycine elution. The max pressure that could be applied was 2500 psi. FIG. 9C shows the linear correlation between monoclonal antibody concentration and average peak area for the glycine elution method. Carryover was >3% for both elution conditions. The lack of hydrophilic layer results in distorted peaks and significant tailing due to nonspecific binding of the monoclonal antibody on the particle surface.

Nonporous Silica Particles with a Diol Coating and a Hydrophilic Coating (Particle Type E)

Silica-based nonporous, epoxy-modified hydrophilic particles were prepared. Nonporous 3.6 micron silica particles were heat treated (900° C.) in air for 10 hours. The surface of the particles was re-hydroxylated using 10% v/v nitric acid at 100° C. for 16 hours. The reaction was then cooled below 40° C. and the particles were isolated via filtration. The product was washed with water until the pH of the filtrate was greater than 5, after which it was washed with acetone 3 times. Isolated particles were dried in a vacuum oven at 80° C. overnight.

The resultant 3.6 micron particles were next coated with glycidoxypropyltrimethoxy silane (GPTMS). 20 mM sodium acetate buffer (pH 5.5) at ˜5 mL/gram particles) was added to a round bottom flask equipped with a thermometer, condenser, and mechanical stirring apparatus. The solution was heated to 70° C., after which GPTMS was added to the flask at a concentration of 10.22 μmol/m² and mixed at temperature. After 1 hour, particles were added to the flask (1 g particle/5 mL of buffer solution). The mixture was stirred at 70° C. for 20 hours, after which the reaction was cooled below 40° C. and filtered. The product was washed 3× with water and transferred to a new flask equipped with a thermometer, condenser, and mechanical stirring apparatus. Acetic acid (0.1M) was added to the flask (5 mL/gram particle) and the slurry was heated to 70° C. for 20 hours. The flask was cooled below 40° C. and filtered. The product was washed with water until the pH of the supernatant increased to above 5 and then washed 3× with methanol. Isolated particles were dried in a vacuum oven at 70° C. for 16 hours.

10 grams of the resultant particles were added into a 1 L three-necked round bottom flask with overhead stirring motor, stirring shift, stir blade, a water-cooled condenser, a nitrogen inlet, and a probe-controlled heating mantle. 100 mL of anhydrous diglyme (diethylene glycol dimethyl ether) was added. The flask was sealed and purged with nitrogen for 15 minutes with moderate stirring. 0.45 g of potassium tert-butoxide was added and the reaction was raised to 70° C. To generate the hydrophilic layer, a mixture of 1.5 g glycidol, 0.4 g glyceroltriglycidyl ether, and 2.2 g of anhydrous diglyme was prepared separately and added to the particle mixture in 4 equal aliquots using 30-minute intervals. The reaction was held at 70° C. for 20 hours, cooled to RT, and filtered. The resultant particles were washed sequentially 6× with water, 3× with methanol, and dried under vacuum overnight at 45° C. The procedure resulted in a hydrophilic layer that is 2-4% by weight of the entire particle.

5 g of the resultant 3.6 micron particles with the hydrophilic coating were added to a mixture of 25 g of ethylene glycol diglycidyl ether (EGDGE) and 25 g of methanol at RT. 0.25 mL of 50% sodium hydroxide in water was added and the reaction was stirred continuously for 20 hours. The particles were isolated by filtration, washed 10× with methanol, and partially dried under nitrogen flow. Particles were stored in a methanol wet bed at 4° C. The resultant particles have sufficient epoxide content to enable functionalized of the particle surface.

Particles were functionalized with Protein A as described in Example 2, resulting in approximately 1.47 ug of Protein A per mg of particle.

The particles were tested for the ability to bind and elute a monoclonal antibody (NISTmAb Standard, available from Sigma-Aldrich). Particles were packed in a 2.1×200 mm column body and connected to an ultra-high performance liquid chromatography system (Acquity Premier available from Waters Technologies Corporation, Milford MA). The column was fast flushed with water (pH 4) to remove unbound protein A at 0.5 mL/min for 10 minutes. The column was then equilibrated in 0.1M sodium phosphate (pH 7.5) at 0.5 mL/min for 10 minutes. 10 uL injections of the monoclonal antibody standard at different concentrations (ranging from 0.001 to 1 ug/uL) was injected onto the column using 0.1M sodium phosphate (pH 7.4) buffer at a flow rate of 1 mL/min. A blank injection of binding buffer was used between samples to calculate carryover. Elution was performed with either 24 mM phosphoric acid (pH 2) or 100 mM glycine (pH 2.4) for 1 minute. The column was equilibrated for 1 minute using 0.1M sodium phosphate buffer (pH 7.4).

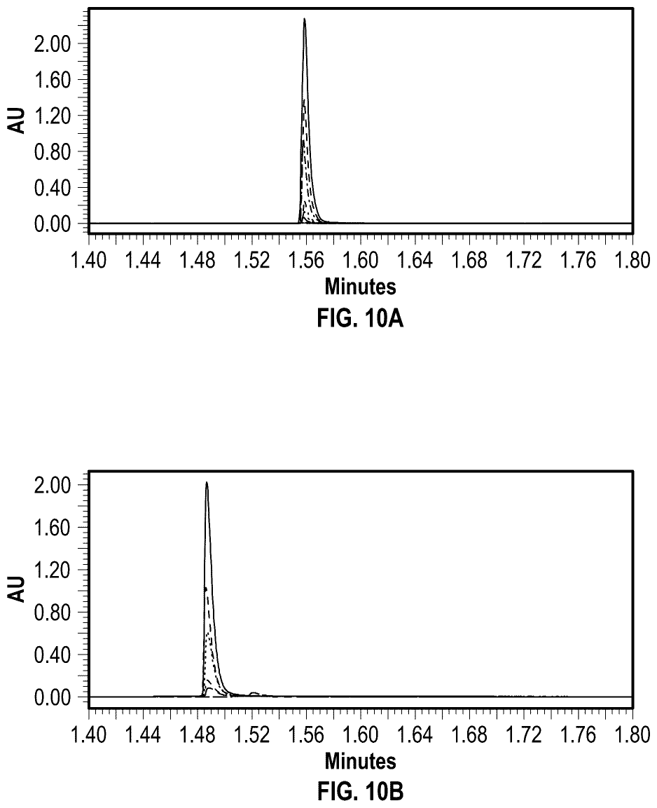

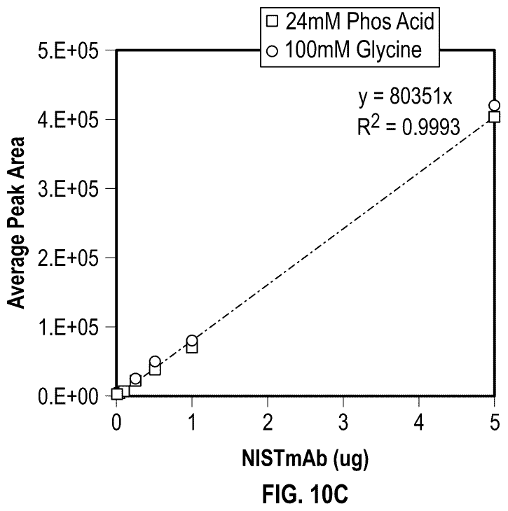

The detection limit was 0.01 ug for the phosphoric acid elution method and 0.25 ug for the glycine elution method. The retention time was shifted in the glycine elution method (see FIG. 10B) as compared to the phosphoric acid elution method (see FIG. 10A). The max pressure that could be applied was 3100 psi. FIG. 10C shows the linear correlation between monoclonal antibody concentration and average peak area for both elution methods. Carryover was less than 7% for both elution methods. The very low amount of protein A immobilized on the surface suggests less epoxide was available for protein A conjugation. The high carryover and significant tailing indicate the hydrophilic layer was not optimal.

In general, silica-based materials are not ideal for affinity chromatography methods due to the tendency of silica to have high levels of non-specific binding. The above particle types (B, D, H, and E) demonstrate that masking of the silica surface with layers of hybrid material, layers of diol, and/or a hydrophilic coating, was not sufficient to eliminate the non-specific binding. Without wishing to be bound by any particular theory, the silica-based particle types might also have lower protein A conjugation efficiency, which can lead to high leaching.

Nonporous Polymer Particles with a Diol Coating and a Hydrophilic Coating (Particle Type C)

Polymer-based nonporous particles were prepared. 65.1 grams of polyvinyl pyrrolidone (PVP-40), 8.9 grams of Triton N-57, 2452.6 grams of reagent alcohol (90% ethanol, 5% methanol, 5% isopropanol), and 153.3 grams of p-xylene were charged into a 4 L cylinder flask reactor equipped with mechanical agitation, condenser, and a thermocouple. The reaction was mixed with agitation at 200 rpm and the dissolved oxygen in the solution was removed by sub-surface purging with nitrogen. After the dissolved oxygen was below 1 ppm, 2.1 grams of azobisisobutyronitrile and 105 grams of divinylbenzene was charged into the reaction. The reaction was raised to 70° C. and held for 20 hours.

1.8 grams of azobisisobutyronitrile was added to the reaction, after which a solution of 67.6 grams of divinylbenzene (DVB) and 20.3 grams of PVP-40 in 202.7 grams of reagent alcohol was metered in via a pump at a constant flow rate over 120 minutes. The reaction was continued at 70° C. for at least 12 hours. The particles were separated from the reaction slurry by filtration and washed sequentially with methanol, tetrahydrofuran (THF), and acetone. The final product was dried in a vacuum oven at 45° C. overnight.

46.2 grams of polymer core was dispersed in 231 grams of ethanol by sonication for 4 minutes using an ultrasonic horn and transferred to a round-bottom flask equipped with mechanical grade agitation, a condenser, and a thermocouple. 49.1 grams of ammonium hydroxide and 462 grams of toluene were added to the reaction and mixed at RT for 60 minutes. The reaction was increased to 35° C. and a mixture of 26.06 grams of TEOS and 66.34 grams of BTEE diluted with 148.9 grams of ethanol and 148.9 grams of toluene were charged via a peristaltic pump at a constant flow rate of 0.95 mL/min. After the reaction, particles were separated by filtration, washed 6× with methanol, and dried in a vacuum oven at 45° C. overnight.

The resultant polymer core particles were dispersed in an aqueous solution of 0.3M tris(hydroxymethyl)aminomethane at a slurry concentration of 5 mL/g. The pH was adjusted to 9.8 using acetic acid. The slurry was then enclosed in a stainless steel autoclave and heated to 155° C. for 20 hours. After cooled to RT, the product was isolated on a 0.5 micron filtration paper and washed with water and methanol. The particles were dried at 80° C. under vacuum for 16 hours.

Following the hydrothermal processing, 50 g of particles were refluxed in 1M hydrochloric acid at a slurry concentration of 10 mL/g for 10 hours. The reaction was cooled to RT and particles isolated via filtration. The particles were washed with water until the pH of the filtrate was greater than 5. The semi-neutralized particles were washed 3× with acetone (10 mL/g). Resultant particles were sized to remove any agglomerates, resulting in a final particle size of 2.3 μm.

The resultant 2.3 micron particles were next coated with glycidoxypropyltrimethoxy silane (GPTMS). 20 mM sodium acetate buffer (pH 5.5) at ˜5 mL/gram particles) was added to a round bottom flask equipped with a thermometer, condenser, and mechanical stirring apparatus. The solution was heated to 70° C., after which GPTMS was added to the flask at a concentration of 10.22 μmol/m² and mixed at temperature. After 1 hour, particles were added to the flask (1 g particle/5 mL of buffer solution). The mixture was stirred at 70° C. for 20 hours, after which the reaction was cooled below 40° C. and filtered. The product was washed 3× with water and transferred to a new flask equipped with a thermometer, condenser, and mechanical stirring apparatus. Acetic acid (0.1M) was added to the flask (5 mL/gram particle) and the slurry was heated to 70° C. for 20 hours. The flask was cooled below 40° C. and filtered. The product was washed with water until the pH of the supernatant increased to above 5 and then washed 3× with methanol. Isolated particles were dried in a vacuum oven at 70° C. for 16 hours.

5 grams of the resultant particles were added into a 1 L three-necked round bottom flask with overhead stirring motor, stirring shift, stir blade, a water-cooled condenser, a nitrogen inlet, and a probe-controlled heating mantle. 50 mL of anhydrous diglyme (diethylene glycol dimethyl ether) was added. The flask was sealed and purged with nitrogen for 15 minutes with moderate stirring. 0.224 g of potassium tert-butoxide was added and the reaction was raised to 70° C. To generate the hydrophilic layer, a mixture of 0.75 g glycidol, 0.19 g glyceroltriglycidyl ether, and 1.1 g of anhydrous diglyme was prepared separately and added to the particle mixture in 4 equal aliquots using 30-minute intervals. The reaction was held at 70° C. for 20 hours, cooled to RT, and filtered. The resultant particles were washed sequentially 6× with water, 3× with methanol, and dried under vacuum overnight at 45° C. The procedure resulted in a hydrophilic layer that is 2-4% by weight of the entire particle.

5 g of the resultant particles with the hydrophilic coating were added to a mixture of 25 g of ethylene glycol diglycidyl ether (EGDGE) and 25 g of methanol at RT. 0.25 mL of 50% sodium hydroxide in water was added and the reaction was stirred continuously for 20 hours. The particles were isolated by filtration, washed 10× with methanol, and partially dried under nitrogen flow. Particles were stored in a methanol wet bed at 4° C. The resultant particles have sufficient epoxide content to enable functionalized of the particle surface.

Particles were functionalized with Protein A as described in Example 2, resulting in approximately 2.5 ug of Protein A per mg of particle.

The particles were tested for the ability to bind and elute a monoclonal antibody (NISTmAb Standard, available from Sigma-Aldrich). Particles were packed in a 2.1×200 mm column body and connected to an ultra-high performance liquid chromatography system (Acquity Premier available from Waters Technologies Corporation, Milford MA). The column was fast flushed with water (pH 4) to remove unbound protein A at 0.5 mL/min for 10 minutes. The column was then equilibrated in 0.1M sodium phosphate (pH 7.5) at 0.5 mL/min for 10 minutes. 10 uL injections of the monoclonal antibody standard at different concentrations (ranging from 0.001 to 1 ug/uL) was injected onto the column using 0.1M sodium phosphate (pH 7.4) buffer at a flow rate of 1 mL/min. A blank injection of binding buffer was used between samples to calculate carryover. Elution was performed with either 24 mM phosphoric acid (pH 2) or 100 mM glycine (pH 2.4) for 1 minute. The column was equilibrated for 1 minute using 0.1M sodium phosphate buffer (pH 7.4).

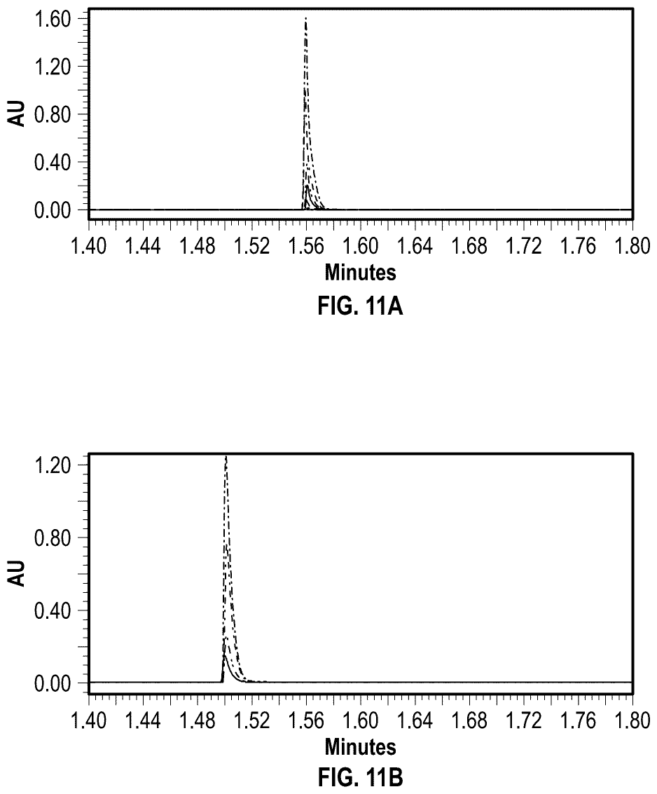

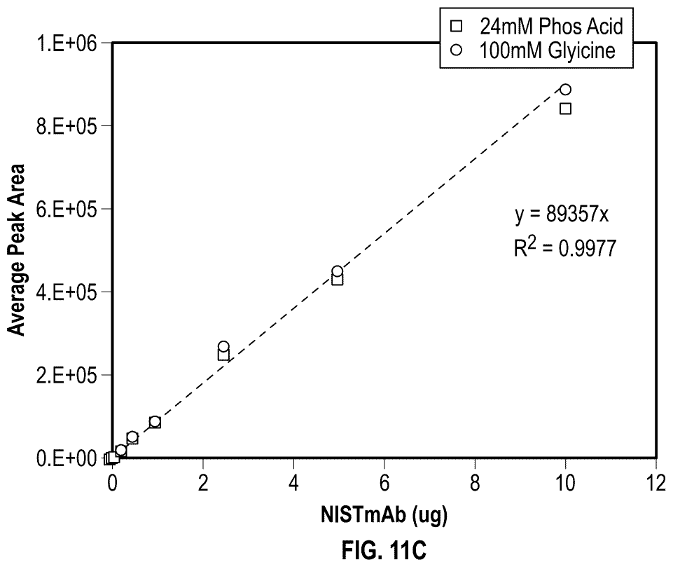

The detection limit was 0.01 ug for the phosphoric acid elution method and 0.25 ug for the glycine elution method. The max pressure that could be applied was 3100 psi. FIG. 11A shows the elution profile for 24 mM phosphoric acid (pH 2) and FIG. 11B shows the elution profile for 100 mM glycine (pH 2.4). FIG. 11C shows the linear correlation between monoclonal antibody concentration and average peak area for both elution methods. Carryover was less than 1% for glycine and 2-10% for phosphoric acid. The hydrophilic layer results in some tailing which is indicative of nonspecific binding of the monoclonal antibody on the particle surface.

Nonporous Polymer Particles with a Diol Coating (Particle Type F)

Polymer-based nonporous particles were prepared. 65.1 grams of polyvinyl pyrrolidone (PVP-40), 8.9 grams of Triton N-57, 2452.6 grams of reagent alcohol (90% ethanol, 5% methanol, 5% isopropanol), and 153.3 grams of p-xylene were charged into a 4 L cylinder flask reactor equipped with mechanical agitation, condenser, and a thermocouple. The reaction was mixed with agitation at 200 rpm and the dissolved oxygen in the solution was removed by sub-surface purging with nitrogen. After the dissolved oxygen was below 1 ppm, 2.1 grams of azobisisobutyronitrile and 105 grams of divinylbenzene was charged into the reaction. The reaction was raised to 70° C. and held for 20 hours.

1.8 grams of azobisisobutyronitrile was added to the reaction, after which a solution of 67.6 grams of divinylbenzene (DVB) and 20.3 grams of PVP-40 in 202.7 grams of reagent alcohol was metered in via a pump at a constant flow rate over 120 minutes. The reaction was continued at 70° C. for at least 12 hours. The particles were separated from the reaction slurry by filtration and washed sequentially with methanol, tetrahydrofuran (THF), and acetone. The final product was dried in a vacuum oven at 45° C. overnight.

46.2 grams of polymer core was dispersed in 231 grams of ethanol by sonication for 4 minutes using an ultrasonic horn and transferred to a round-bottom flask equipped with mechanical grade agitation, a condenser, and a thermocouple. 49.1 grams of ammonium hydroxide and 462 grams of toluene were added to the reaction and mixed at RT for 60 minutes. The reaction was increased to 35° C. and a mixture of 26.06 grams of TEOS and 66.34 grams of BTEE diluted with 148.9 grams of ethanol and 148.9 grams of toluene were charged via a peristaltic pump at a constant flow rate of 0.95 mL/min. After the reaction, particles were separated by filtration, washed 6× with methanol, and dried in a vacuum oven at 45° C. overnight.

The resultant polymer core particles were dispersed in an aqueous solution of 0.3M tris(hydroxymethyl)aminomethane at a slurry concentration of 5 mL/g. The pH was adjusted to 9.8 using acetic acid. The slurry was then enclosed in a stainless steel autoclave and heated to 155° C. for 20 hours. After cooled to RT, the product was isolated on a 0.5 micron filtration paper and washed with water and methanol. The particles were dried at 80° C. under vacuum for 16 hours.

Following the hydrothermal processing, 50 g of particles were refluxed in 1M hydrochloric acid at a slurry concentration of 10 mL/g for 10 hours. The reaction was cooled to RT and particles isolated via filtration. The particles were washed with water until the pH of the filtrate was greater than 5. The semi-neutralized particles were washed 3× with acetone (10 mL/g). Resultant particles were sized to remove any agglomerates, resulting in a final particle size of 2.3 μm.

The resultant 2.3 micron particles were next coated with glycidoxypropyltrimethoxy silane (GPTMS). 20 mM sodium acetate buffer (pH 5.5) at ˜5 mL/gram particles) was added to a round bottom flask equipped with a thermometer, condenser, and mechanical stirring apparatus. The solution was heated to 70° C., after which GPTMS was added to the flask at a concentration of 10.22 μmol/m² and mixed at temperature. After 1 hour, particles were added to the flask (1 g particle/5 mL of buffer solution). The mixture was stirred at 70° C. for 20 hours, after which the reaction was cooled below 40° C. and filtered. The product was washed 3× with water and transferred to a new flask equipped with a thermometer, condenser, and mechanical stirring apparatus. Acetic acid (0.1M) was added to the flask (5 mL/gram particle) and the slurry was heated to 70° C. for 20 hours. The flask was cooled below 40° C. and filtered. The product was washed with water until the pH of the supernatant increased to above 5 and then washed 3× with methanol. Isolated particles were dried in a vacuum oven at 70° C. for 16 hours.

5 g of the resultant particles with the hydrophilic coating were added to a mixture of 25 g of ethylene glycol diglycidyl ether (EGDGE) and 25 g of methanol at RT. 0.25 mL of 50% sodium hydroxide in water was added and the reaction was stirred continuously for 20 hours. The particles were isolated by filtration, washed 10× with methanol, and partially dried under nitrogen flow. Particles were stored in a methanol wet bed at 4° C. The resultant particles have sufficient epoxide content to enable functionalized of the particle surface.

Particles were functionalized with Protein A as described in Example 2, resulting in approximately 4.66 ug of Protein A per mg of particle.

The particles were tested for the ability to bind and elute a monoclonal antibody (NISTmAb Standard, available from Sigma-Aldrich). Particles were packed in a 2.1×200 mm column body and connected to an ultra-high performance liquid chromatography system (Acquity Premier available from Waters Technologies Corporation, Milford MA). The column was fast flushed with water (pH 4) to remove unbound protein A at 0.5 mL/min for 10 minutes. The column was then equilibrated in 0.1M sodium phosphate (pH 7.5) at 0.5 mL/min for 10 minutes. 10 uL injections of the monoclonal antibody standard at different concentrations (ranging from 0.001 to 1 ug/uL) was injected onto the column using 0.1M sodium phosphate (pH 7.4) buffer at a flow rate of 1 mL/min. A blank injection of binding buffer was used between samples to calculate carryover. Elution was performed with either 24 mM phosphoric acid (pH 2) or 100 mM glycine (pH 2.4) for 1 minute. The column was equilibrated for 1 minute using 0.1M sodium phosphate buffer (pH 7.4).

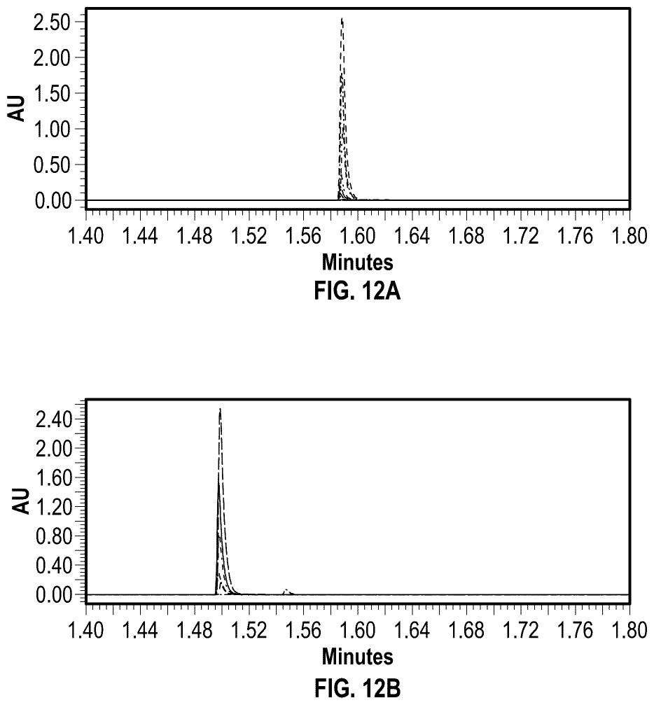

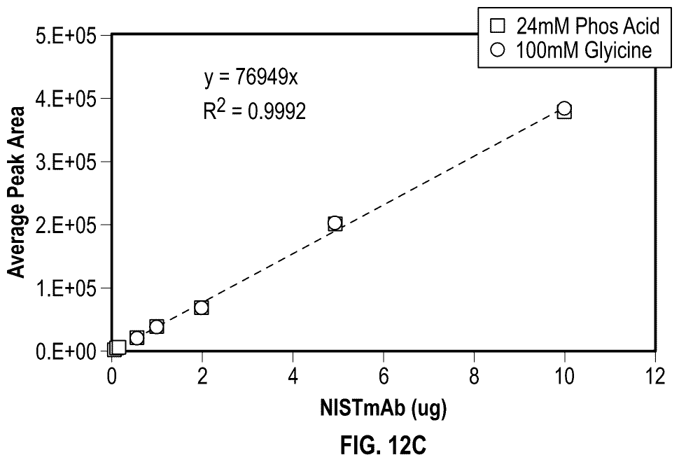

The detection limit was 0.01 ug for the phosphoric acid elution method and 0.25 ug for the glycine elution method. The retention time was shifted at the lower concentrations for glycine elution (see FIG. 12B), which was not observed in the phosphoric acid elution method (see FIG. 12A). The max pressure that could be applied was 4900 psi. FIG. 12C shows the linear correlation between monoclonal antibody concentration and average peak area for both elution methods. Carryover was less than 1.5% for glycine and less than 6% for phosphoric acid. There was low carryover observed, but significant tailing due to nonspecific binding of the monoclonal antibody on the particle surface.

Particles of Example 1 Functionalized with Protein A (Particle Type A)

As a comparison, the particles of Examples 1-2 were tested the same bind and elute method. Particles were packed in a 2.1×200 mm column body and connected to an ultra-high performance liquid chromatography system (Acquity Premier available from Waters Technologies Corporation, Milford MA). The column was fast flushed with water (pH 4) to remove unbound protein A at 0.5 mL/min for 10 minutes. The column was then equilibrated in 0.1M sodium phosphate (pH 7.5) at 0.5 mL/min for 10 minutes. 10 uL injections of the monoclonal antibody standard at different concentrations (ranging from 0.001 to 1 ug/uL) was injected onto the column using 0.1M sodium phosphate (pH 7.4) buffer at a flow rate of 1 mL/min. A blank injection of binding buffer was used between samples to calculate carryover. Elution was performed with either 24 mM phosphoric acid (pH 2) or 100 mM glycine (pH 2.4) for 1 minute. The column was equilibrated for 1 minute using 0.1M sodium phosphate buffer (pH 7.4).

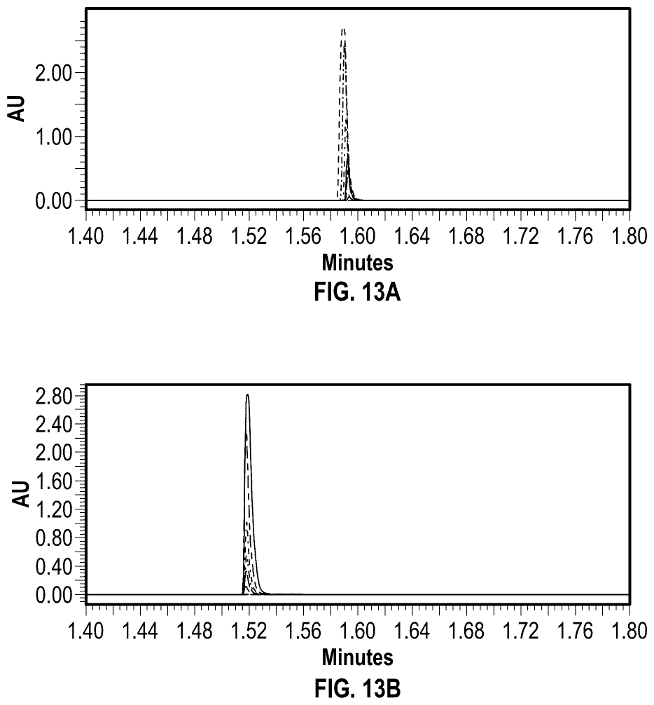

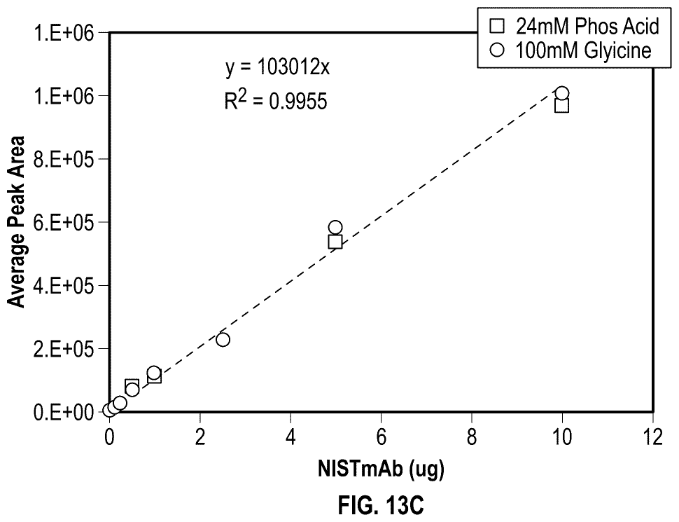

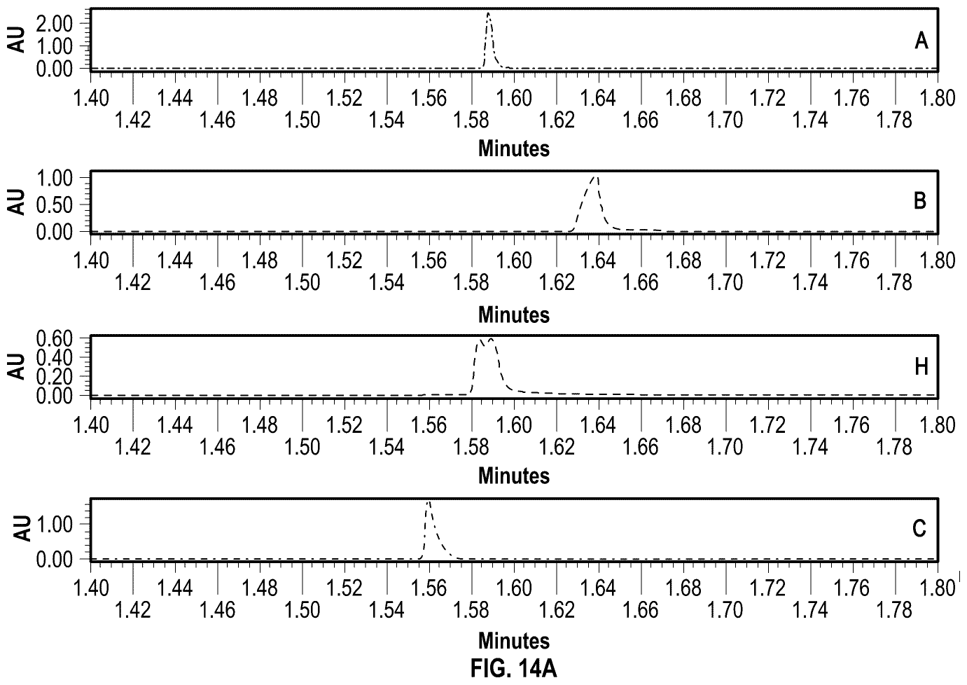

The detection limit was 0.01 ug for the phosphoric acid elution method and 0.1 ug for the glycine elution method. The max pressure that could be applied was 5000 psi. Carryover was less than 2% for glycine and less than 6% for phosphoric acid. High sensitivity and detector saturation (10 ug) was observed with low amounts of monoclonal antibody as shown in FIG. 13A (24 mM phosphoric acid (pH 2)) and FIG. 13B (100 mM glycine (pH 2.4)). FIG. 13C shows the linear correlation between monoclonal antibody concentration and average peak area.

### Comparisons Between Particle Types A-H

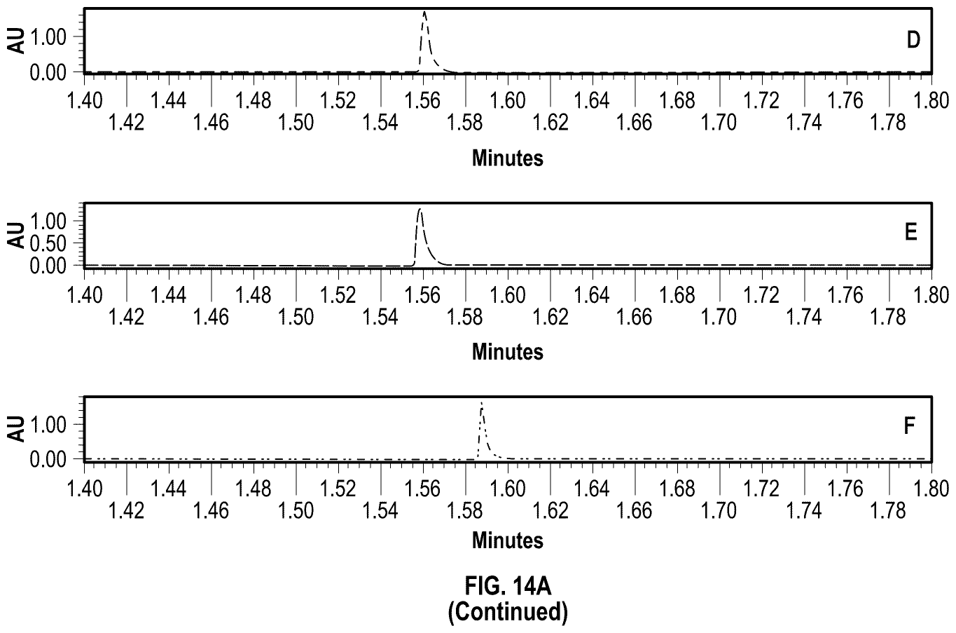

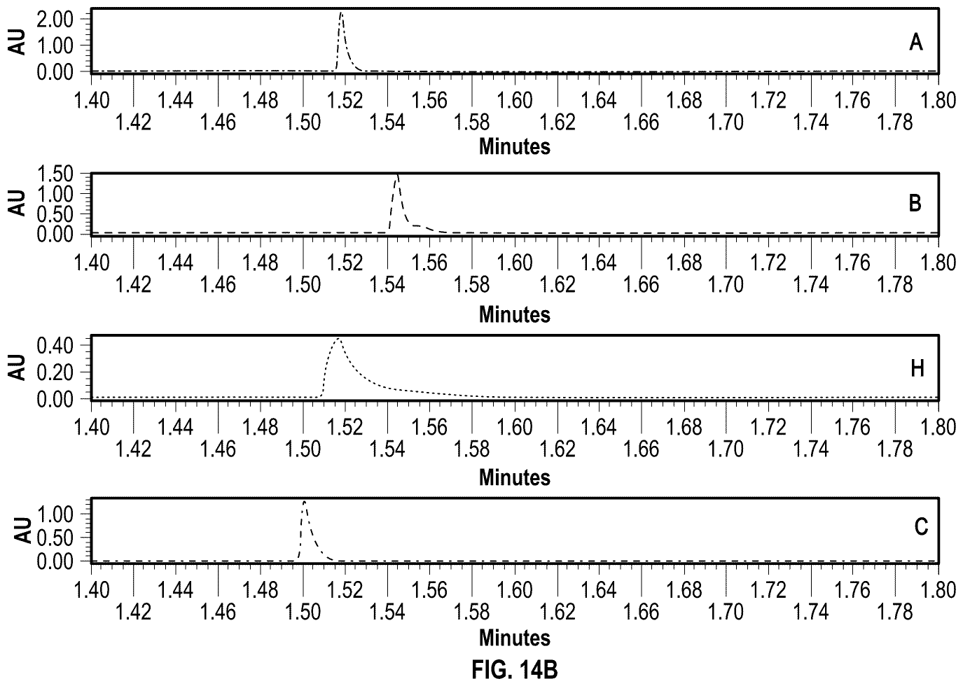

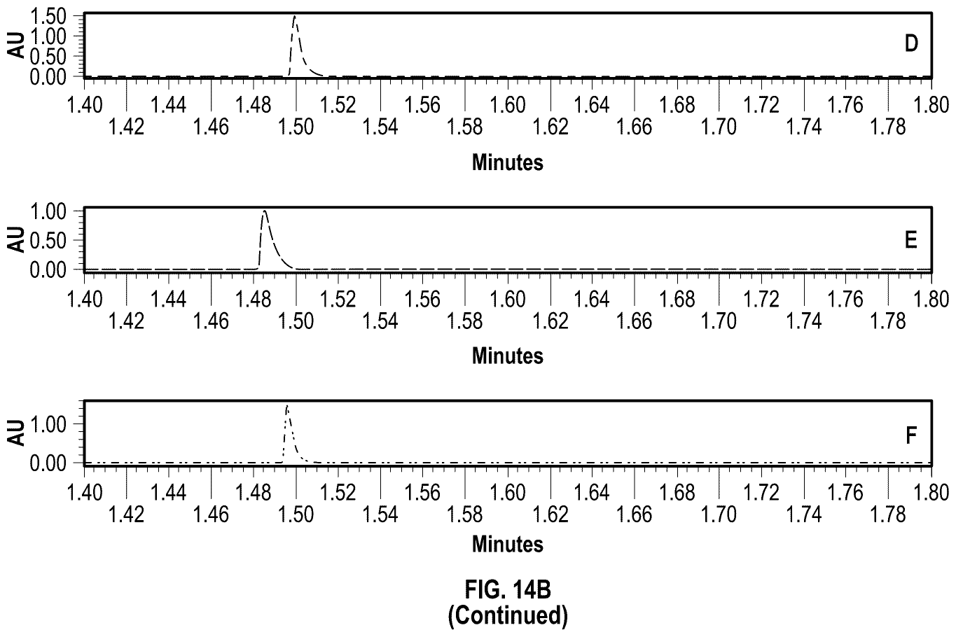

FIG. 14A-14B provide comparisons of peak shape at the highest concentration of tested sample (5 ug of monoclonal antibody) using the 24 mM phosphoric acid elution method (FIG. 14A) and the 100 mM glycine elution method (FIG. 14B). As shown in FIG. 14A, with the phosphoric acid elution method, particle types B, C, D, and E exhibited a shift in retention along with peak tailing, while particle type H resulted in peak splitting. Similarly, as shown in FIG. 14B, particle types B and C, D, E, and F exhibited a shift in retention. Particle types B, H, C, D, and E also had broadened peaks with peak tailing. Notably, particle type A resulted in narrow peaks with higher intensity relative to other materials tested.

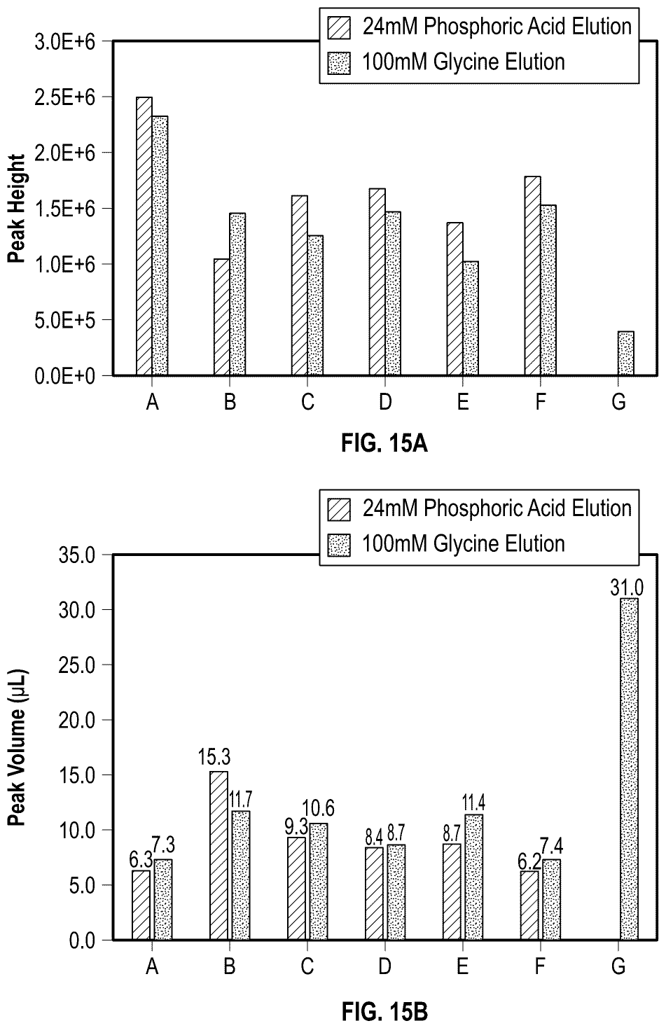

FIG. 15A shows the peak height for particle types A, B, C, D, E, F, and G (porous particles as described in Example 4). FIG. 15B shows the peak volume for said particles. As shown in FIG. 15A, particle type A has >1.4× peak height compared to all other particle types tested for both elution methods. Particle types A, C, D, E, and F had similar, small peak volumes. Without being bound by theory, the low peak volume may be due in part to the small nonporous particles having increased kinetics. In contrast, particle type G had significantly higher peak volumes—almost 4× more than that of particle type A.

What is claimed is:

1. A chromatographic column comprising:
a column body formed of a metal or a metal alloy, the column body housing a plurality of particles, each particle of the plurality of particles comprising:
a nonporous polymer core;
a hydrophilic surface on an outer layer of the nonporous polymer core; and
one or more molecules of an immunoglobulin-binding protein conjugated to the hydrophilic surface,
wherein the particle has an average particle size between 1.5 μm to 8 μm.

2. The particle of claim 1, wherein the immunoglobulin-binding protein is selected from the group consisting of: Protein A, Protein G, Protein A/G, Protein L, or a binding domain thereof.

3. The particle of claim 1, wherein the nonporous polymer core has a gradient composition.

4. The particle of claim 1, wherein the nonporous polymer core comprises divinylbenzene (80%).

5. The particle of claim 1, wherein the hydrophilic surface is selected from the group consisting of: (3-glycidyloxypropyl)trimethoxysilane, (3-glycidyloxypropyl)triethoxysilane, polyacrylate, glycidol, glycerol triglycidyl ether, butyl diglycidol ether, and poly(methyl acrylate).

6. The particle of claim 1, wherein the one or more molecules of immunoglobulin-binding protein are conjugated to the hydrophilic surface of the particle via an epoxy linker.

7. The particle of claim 1, wherein the average particle size is between 2 μm to 5 μm.

8. The particle of claim 7, wherein the average particle size is 3.5 μm.

9. The particle of claim 2, wherein the immunoglobulin-binding protein has a surface coverage concentration of between 3-9 μg immunoglobulin-binding protein per mg of particle.

10. The particle of claim 9, wherein the immunoglobulin-binding protein is Protein A.

11. The chromatographic column of claim 1, wherein at least a portion of an interior surface of the column body is coated with an alkylsilyl material.

12. The chromatographic column of claim 11, further comprising frits within the column body, wherein the frits are coated with the alkylsilyl material.

13. The chromatographic column of claim 11, wherein the alkylsilyl material is a hydrophilic, non-ionic layer of polyethylene glycol silane.

14. A chromatographic device comprising:
the chromatographic column of claim 1,
a column injector positioned upstream of the chromatographic column, and tubing in fluidic connection with and located downstream of the chromatographic column,
wherein a portion of an internal surface of the column injector and a portion of an internal surface of the tubing are coated with an alkylsilyl material.

15. A method for enriching an immunoglobulin, the method comprising:
a) selecting a chromatographic column, wherein the chromatographic column comprises a column body formed of a metal or a metal alloy, the column body housing a plurality of particles, each particle of the plurality of particles comprising:
a nonporous polymer core;
a hydrophilic surface on an outer layer of the nonporous polymer core; and
one or more molecules of an immunoglobulin-binding protein conjugated to the hydrophilic surface,
wherein the particle has an average particle size between 1.5 μm to 8 μm;
b) washing the chromatographic column with a binding buffer;
c) applying a solution containing an immunoglobulin to the chromatographic column; and
d) washing the chromatographic column with an elution buffer such that the immunoglobulin is eluted from the chromatographic column.

16-31. (canceled)
